SEQUENCE LISTING (8 sequences)

SEQ ID NO: 1 - protein, 134 aa, Homo sapiens
SPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 2 - DNA, 650 bp, Mus musculus
  IgSP_ORF1 (108)..(153)
  IgSP-GDNF_ORF2 (246)..(649)
tcttctttttcctacagctcctgggcaacgtgctggttattgtgctgtctcatcattttggcaaagaattctgcagtcgacggtaccgcgggcccgggatccgccaccatgaagtgcagctgggttatcttcttcctgatggcagtggttacaggtaaggggctcccaagtcccaaacttgagggtccataaactctgtgacagtggcaatcactttgcctttctttctacaggggtgaattcgtcaccagataaacaaatggcagtgctgcccagacgggagagaaacagacaggccgctgccgccaaccctgagaacagccggggcaagggcagaagaggccagcggggcaagaacagaggctgcgtgctgaccgccatccacctgaacgtgaccgacctgggcctgggctacgagacaaaagaggaactgatcttccggtactgcagcggctcttgcgacgccgccgagacaacctacgacaagatcctgaagaacctgagccggaacagacggctggtgtccgacaaagtgggccaggcctgctgcagacctatcgccttcgacgacgacctgagcttcctggacgacaacctggtgtaccacatcctgcggaagcacagcgccaagagatgcggctgcatctgac

SEQ ID NO: 3 - DNA, 639 bp, Homo sapiens
atgaagctgtgggacgtggtggccgtgtgcctggtgctgctgcacaccgccagcgccttccctctgcctgccggaaagcggcctcctgaggctcctgccgaggacagaagcctgggcaggcggagagcccccttcgccctgagcagcgacagcaacatgcccgaggactaccccgaccagttcgacgacgtgatggacttcatccaggccaccatcaagcggctgaagcggagccccgacaagcagatggccgtgctgcccagacgggagcggaaccggcaggccgctgccgccaaccccgagaacagccggggcaagggcagacggggccagcggggcaagaaccggggctgcgtgctgaccgccatccacctgaacgtgaccgacctgggcctgggctacgagacaaaagaggaactgatcttccggtactgcagcggcagctgcgacgccgccgagacaacctacgacaagatcctgaagaacctgagccggaaccggcggctggtgtccgacaaagtgggccaggcctgctgtcggcctatcgccttcgacgacgacctgagcttcctggacgacaacctggtgtaccacatcctgcggaagcacagcgccaagagatgcggctgcatctgatga

SEQ ID NO: 4 - protein, 211 aa, Homo sapiens
MKLWDVVAVCLVLLHTASAFPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 5 - protein, 134 aa, Mus musculus
SPDKQAAALPRRERNRQAAAASPENSRGKGRRGQRGKNRGCVLTAIHLNVTDLGLGYETKEELIFRYCSGSCESAETMYDKILKNLSRSRRLTSDKVGQACCRPVAFDDDLSFLDDNLVYHILRKHSAKRCGCI

SEQ ID NO: 6 - protein, 133 aa, Rattus norvegicus
SPDKQAAALPRRERNRQAAAASPENSRGKGRRGQRGKNRGCVLTAIHLNVTDLGLGYETKEEIFRYCSGSCEAAETMYDKILKNLSRSRRLTSDKVGQACCRPVAFDDDLSFLDDSLVYHILRKHSAKRCGCI

SEQ ID NO: 7 - DNA, 7832 bp, Artifical Sequence
  source (1)..(7832): Co-transfection of ARPE-19 cells with plasmids pT2.CAn.hoG
caaggcgattaagttgggtaacgccagggttttcccagtcacgacgttgtaaaacgacggccagtgagcgcgcgtaatacgactcactatagggcgaattggagctcggatccctatacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatgtcatggctttagaagctagatctagagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattctgcagtcgacggtaccgcgggcccgggatccgccaccatgaagtgcagctgggttatcttcttcctgatggcagtggttacaggtaaggggctcccaagtcccaaacttgagggtccataaactctgtgacagtggcaatcactttgcctttctttctacaggggtgaattcgtcaccagataaacaaatggcagtgctgcccagacgggagagaaacagacaggccgctgccgccaaccctgagaacagccggggcaagggcagaagaggccagcggggcaagaacagaggctgcgtgctgaccgccatccacctgaacgtgaccgacctgggcctgggctacgagacaaaagaggaactgatcttccggtactgcagcggctcttgcgacgccgccgagacaacctacgacaagatcctgaagaacctgagccggaacagacggctggtgtccgacaaagtgggccaggcctgctgcagacctatcgccttcgacgacgacctgagcttcctggacgacaacctggtgtaccacatcctgcggaagcacagcgccaagagatgcggctgcatctgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggtgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtagatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggaatcaagcttgtggaaggctactcgaaatgtttgacccaagttaaacaatttaaaggcaatgctaccaaatactaattgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactgtatagggatcctctagctagagtcgacctcgagggggggcccggtacccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctg

SEQ ID NO: 8 - DNA, 7930 bp, Artifical Sequence
  source (1)..(7930): Co-trnasfections of ARPE-19 cells with plasmids pCMV-SB-100x
caaggcgattaagttgggtaacgccagggttttcccagtcacgacgttgtaaaacgacggccagtgagcgcgcgtaatacgactcactatagggcgaattggagctcggatccctatacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatgtcatggctttagaagctagatctagagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattctgcagtcgacggtaccgcgggcccgggatccgccaccatgaagctgtgggacgtggtggccgtgtgcctggtgctgctgcacaccgccagcgccttccctctgcctgccggaaagcggcctcctgaggctcctgccgaggacagaagcctgggcaggcggagagcccccttcgccctgagcagcgacagcaacatgcccgaggactaccccgaccagttcgacgacgtgatggacttcatccaggccaccatcaagcggctgaagcggagccccgacaagcagatggccgtgctgcccagacgggagcggaaccggcaggccgctgccgccaaccccgagaacagccggggcaagggcagacggggccagcggggcaagaaccggggctgcgtgctgaccgccatccacctgaacgtgaccgacctgggcctgggctacgagacaaaagaggaactgatcttccggtactgcagcggcagctgcgacgccgccgagacaacctacgacaagatcctgaagaacctgagccggaaccggcggctggtgtccgacaaagtgggccaggcctgctgtcggcctatcgccttcgacgacgacctgagcttcctggacgacaacctggtgtaccacatcctgcggaagcacagcgccaagagatgcggctgcatctgatgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggtgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtagatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggaatcaagcttgtggaaggctactcgaaatgtttgacccaagttaaacaatttaaaggcaatgctaccaaatactaattgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactgtatagggatcctctagctagagtcgacctcgagggggggcccggtacccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctg

(19) United States
(12) Patent Application Publication
(10) Pub. No.: US 2023/0047254 A1
(43) Pub. Date: Feb. 16, 2023

(54) Mammalian Cells Secreting GDNF and Their Therapeutic Use

(71) Applicant: Gloriana Therapeutics, Warren, RI (US)

(72) Inventor: LARS U. WAHLBERG, Warren, RI (US)

(21) Appl. No.: 17/789,653

(22) PCT Filed: Dec. 29, 2019
(86) PCT No.: PCT/US2020/067351

§ 371 Date: Jun. 28, 2022

Related U.S. Application Data
(60) Provisional application No. 62/954,640, filed on Dec. 29, 2019.
(60) Provisional application No. 63/131,304, filed on Dec. 28, 2020.

(51) Int. Cl. A61L 27/38; C12N 5/07; A61K 48/00; A61L 27/54

(52) U.S. Cl. CPC A61L 27/383; C12N 5/06; A61K 48/00; A61L 27/54; A61L 2300/414; A61L 2300/64

(57) ABSTRACT

The present invention concerns methods and compositions for gene therapy, in particular in vivo gene therapy for delivery of bioactive glial derived neurotrophic factor (GDNF) for the treatment of Parkinson's Disease. The invention also concerns mammalian cells capable of producing GDNF in increased amounts as well as the use of these cells for recombinant production of bioactive GDNF and for therapeutic use. The invention also includes a device that may be implanted in the cochlear of a patient that is capable of secreting GDNF.

### STATEMENT REGARDING SEQUENCE LISTING

The Sequence Listing associated with this application is provided in text format in lieu of a paper copy and is hereby incorporated by reference into the specification. The name of the text file containing the Sequence Listing is P8881US00_Gloriana_ARPE-19_Cell_line_ST25.txt. The text file is 28 KB, was created on Nov. 19, 2019, and is being submitted electronically via EFS-Web.

### FIELD OF INVENTION

The present invention concerns methods and compositions for gene therapy, in particular in vivo gene therapy for delivery of bioactive glial cell line-derived neurotrophic factor (GDNF) for the treatment of Parkinson's Disease. In another aspect the invention relates to expression constructs comprising a codon-optimized version of a full length human GDNF sequence. The invention also concerns mammalian cells capable of producing GDNF in increased amounts as well as the use of these cells for recombinant production of bioactive GDNF and for therapeutic use.

### BACKGROUND OF THE INVENTION

Parkinson's disease (PD) is a devastating neurodegenerative disorder that afflicts between 1 and 1.5 million Americans. Over 35,000 new cases are diagnosed each year. The incidence of Parkinson's disease is highest in the over-50 age group, although an alarming number of new cases have been reported in younger patients.

The cardinal features of Parkinson's disease are slowness of movement (bradykinesia), a tremor or trembling in the hands, arms, legs, jaw, and face, stiffness of the limbs and trunk, and postural instability. As these symptoms progress, patients may experience difficulty walking, talking, or completing other simple daily living tasks. These behavioral deficits are linked to the degeneration of the nigrostriatal system in the brain, which is responsible for the production of smooth, purposeful movements. Specifically, nerve cells located in the substantia nigra degenerate and there is an accompanying loss of dopamine that is made by these cells. The substantia nigra nerve cells extend axons or processes to the striatum, where the dopamine is secreted and utilized. It has been estimated that an 80% loss of dopamine within the striatum needs to occur before the symptoms of PD emerge.

At present, levodopa (trade name Sinemet) is the mainstay treatment for Parkinson's disease. In the brain, levadopa is converted to dopamine, which corrects the dopamine deficiency in the brains of patients with Parkinson's disease. When levodopa is administered in combination with the peripheral decarboxylase inhibitor carbidopa, PD patients experience dramatic benefits. The problem, however, is that while levodopa therapy diminishes the symptoms of PD, it does not replace lost nerve cells and does not halt the progression of the disease. As PD progresses, patients require increasing doses of levodopa and side effects, most notably disabling involuntary movements and rigidity, may emerge. In fact, movement disorder specialists often delay the use of levodopa and initially use other dopaminergic drugs in order to save the use of levodopa for later on in the disease process when patients need it the most.

Thus, levodopa has its limitations and additional therapeutic strategies for Parkinson's disease need to be established. In this regard, interest in surgical treatments for PD has been rekindled. Recently, a procedure called deep brain stimulation has gained considerable attention. In this procedure, electrodes are placed in brain regions that are overactive in PD so that electrical stimulation of these brain regions corrects the overactivity. In some patients, dramatic benefits can be achieved. Other surgical interventions are aimed at improving the function of the nigrostriatal system. Transplants of dopaminergic cells have been successful in ameliorating motor deficits in animal models of Parkinson's disease. Initial clinical trials of dopaminergic cell transplantation in humans have been successful, while a single double blind clinical trial revealed benefit in younger, but not older patients. However, some of the patients receiving grafts developed disabling involuntary movements. Thus, at present, cellular transplants should still be considered an experimental approach.

Another approach aims to deliver growth factors into the nigrostriatal system in an attempt to prevent the degeneration of substantia nigra neurons and the accompanying loss of the neurotransmitter dopamine.

Many laboratories across the world have demonstrated that Glial Cell-line Derived Neurotrophic Factor (GDNF) can prevent the structural and functional consequences of degeneration of the nigrostriatal system in studies conducted in rats and primates. Delivery of GDNF to the CNS has been achieved in pre-clinical studies using protein injections, delivery via pumps and by in vivo gene therapy. Numerous studies describe transduction of CNS cells using AAV or lentivirus expressing GDNF (Kordower, (2003), Ann Neurol, 53 (suppl 3):s120-s34; WO 03/018821, Ozawa et al.; US-2002/187951, Aebischer et al.; Georgievska et al., (2002), Exp Nerol 117(2):461-74; Georgievska et al., (2002), NeuroReport 13(1):75-82; Wang et al., (2002), Gene Thera, 9(6):381-9; US-2002/031493, Rohne-Poulenc Rorer S A; U.S. Pat. No. 6,180,613, Roeckefeller University; Kozlowski et al., (2000), Exp Neurol, 166(1):1-15; Bensadoun, (2000), Exp Neurol, 164(1):15-24; Connor et al., (1999), Gene Therapy, 6(12):1936-51; Mandel et al., (1997), PNAS, 94(25):14083-8; Lapchak et al., (1997), Brain Res, 777(1,2):153-60; Bilang-Bleuel et al., (1997), PNAS 94(16):8818-23).

Other in vivo gene therapy approaches to the treatment of Parkinson's Disease include transduction with virus expressing aromatic L-amino acid decarboxylase (AADC), suthalamic glutamic acid decarboxylase (GAD) (Marutso, (2003), Nippon Naika Gakkai Zasshi, 92(8):1461-6; Howard, (2003), Nat Biotechnol, 21(10):117-8).

Although GDNF seems to be a promising candidate for treatment of Parkinson's Disease in human beings, GDNF treatment is reported to result in certain side-effects, mainly weight-loss and allodynia (Hoane et al., (1999), 160(1):235-43). There is, therefore, a need in the art for developing alternative strategies for treatment of Parkinson's Disease in particular strategies that aim at preventing the degeneration of substantia nigra neurons.

### SUMMARY OF THE INVENTION

The present inventors have performed a series of pre-clinical animal studies based on delivery of GDNF family growth factors to the striatum with human ARPE-19 cell derived clones secreting high levels of GDNF in a 6-OHDA lesion model. The 6-OHDA lesion model is a well-known animal model for Parkinson's Disease. These experiments have surprisingly shown that implantation of cells secreting high levels of GDNF exhibited a neuroprotective benefit, and said cells continued to secret GDNF for an extended period of time. Secreted GDNF was correctly processed by the surrounding brain tissue.

Consequently, in a first aspect the invention relates to a method for treatment of Parkinson's Disease, said method comprising administering to the central nervous system of an individual in need thereof a therapeutically effective amount of cells containing an expression vector, said vector comprising a promoter sequence capable of directing the expression of an operably linked polypeptide, said polypeptide comprising a signal peptide capable of functioning in a mammalian cell, and a human, murine or rat GDNF, selected from the group consisting of pro-GDNF, mature GDNF, N-terminally truncated mature GDNF, and a sequence variant of any such GDNF.

In a preferred embodiment, cell lines of the invention were created by the co-transfection of ARPE-19 cells with plasmids pT2.CAn.hoG (SEQ ID No. 7) and pCMV-SB-100x (SEQ ID No. 8). The latter plasmid expresses a hyperactive version of the Sleeping Beauty transposase. The plasmid does not contain a eukaryotic selection marker cassette and is, thus, intentionally only transiently expressed. The transfected cells were then screened for high, stable levels of mature GDNF expression.

One important advantage of using the high efficiency expression constructs described in the present invention is that the therapeutic benefit of GDNF can be received using fewer cells and fewer insertions into the patient.

In another aspect, the invention relates to use of a cell expression vector, said vector comprising a promoter sequence capable of directing the expression of an operably linked polypeptide, said polypeptide comprising a signal peptide capable of functioning in a mammalian cell, and a human, murine or rat GDNF selected from the group consisting of pro-GDNF, mature GDNF, N-terminally truncated mature GDNF, and a sequence variant of any such GDNF; for the preparation of a medicament for the treatment of Parkinson's Disease.

In a further aspect, the invention relates to a pharmaceutical composition comprising the vector according to the invention and one or more pharmaceutically acceptable adjuvants, excipients, carriers and/or diluents. The pharmaceutical composition can be used for in vivo and ex vivo gene therapy.

In a further aspect, the invention relates to an isolated host cell transduced with the vector according to the invention.

Such transduced host cells have turned out to produce unexpected high amounts of GDNF compared known to GDNF-producing cells and compared to cells transduced with viral vectors encoding GDNF. The transduced host cells of the present invention therefore constitute a promising source of cells for the industrial scale production of GDNF.

In a further aspect, the invention relates to a chimeric non-human mammal comprising at least one cell being transduced with the vector according to the invention. Such animals which overexpress GDNF can be used for gene profiling and in the screening and development of drugs.

Preferably the transduced cell has the genotype of the individual animal, i.e., is not an allogeneic or xenogeneic transplant.

In a further aspect, the invention relates to an implantable cell culture device, the device comprising:
a semipermeable membrane permitting the diffusion of GDNF therethrough; and
at least one isolated host cell according to the invention.

These capsules can be used for the local delivery of GDNF upon transplantation into the central nervous system. Localized and prolonged delivery of growth factor is a preferred administration method for the treatment of a number of CNS disorders, including but not limited to Parkinson's disease, Alzheimer's disease, Huntington's disease, stroke, and amyotrophic lateral sclerosis (ALS).

In a further aspect, the invention relates to a biocompatible capsule comprising: a core comprising living packaging cells that secrete a viral vector for infection of a target cell, wherein the viral vector is a vector according to the invention; and an external jacket surrounding said core, said jacket comprising a permeable biocompatible material, said material having a porosity selected to permit passage of retroviral vectors of approximately 100 nm diameter thereacross, permitting release of said viral vector from said capsule.

The capsules of this invention provide for the delivery of viral particles to a desired site in a patient using a capsular approach. Encapsulation of vector-producing cell lines permits continuous delivery of the viral particle to the target site, as opposed to a single infusion. In addition, repeat therapy is possible, with reduced likelihood of immune attack. The capsules have pores large enough to allow passage of viral particles released from the packaging cells, yet prevent host-cell passage into the capsule.

This capsular approach increases the safety and control of the therapy because the devices can easily be retrieved (terminating the treatment) or explanted and re-implanted (modifying the treatment). Further, the chance of infection is reduced because the capsular device is not open or externalized.

Finally, because encapsulation prevents the packaging cells from migrating within the patient and prolongs the viability of the packaging cells upon implant, fewer cells are likely to be needed for this therapy. This may be advantageous in further lowering an immune reaction in the patient.

In a further aspect, the invention relates to use of the vector according to the invention as a medicament.

In a still further aspect, the invention relates to use of the vector according to the invention for the preparation of a medicament for the treatment of a nervous system disorder.

In another aspect, the invention relates to the use of the vector according to the invention for the preparation of a medicament for the treatment of a CNS disorder.

Furthermore, the invention relates to a method of treating a nervous system disease, said method comprising administering to an individual in need thereof: a therapeutically effective amount of the vector of the invention; or a therapeutically effective amount of the pharmaceutical composition of the invention; or a biocompatible device comprising a packaging cell line according to the invention.

According to this aspect of the invention there is provided improved in vivo gene therapy methods for the treatment of nervous system diseases. As evidenced by the appended examples, in vivo transduction with the vectors of the present invention results in hitherto unseen secretion and tissue distribution of the encoded therapeutic factors, e.g., GDNF, and as a consequence improved therapeutic effect.

In a still further aspect, the invention relates to a method of treating a nervous system disease, said method comprising transplanting to an individual in need thereof:
i. a therapeutically effective amount of the transduced cells of the invention; or
ii. an implantable device according to the invention.

This aspect provides another way of treating nervous system disorders based on ex vivo gene therapy and implantation of therapeutic cells capable of secreting increased amounts of the GDNF.

In a further aspect, the invention relates to a mammalian cell capable of secreting GDNF or a functional equivalent thereof in amounts in excess of 20 μg GDNF/10⁵ cells/24 hrs for more than 6 months.

The GDNF-producing cells described in the present invention produce GDNF in amounts exceeding that seen in prior art mammalian cells by at least one order of magnitude. The GDNF producing cells of the present invention make it feasible to produce the protein in fermenters using mammalian cells with the advantage that the protein is correctly processed, glycosylated, and folded and can be recovered easily from the culture medium.

### BRIEF DESCRIPTION OF THE DRAWINGS

FIG. 1 describes plasmid maps of the GDNF expression vectors used to create GDNF secreting cell clones: pT2.CAn.hoG (A) and pT2.CAn.hoIgSP.GDNF (B).

FIG. 2 describes GDNF ELISA results for a selection of the best GDNF clones in 2D confluent culture.

FIG. 3 describes a GDNF Western blot of conditioned media samples from clone CA-9, ARPE-19/pT2.CAn.ho.IgSP.GDNF #2 (IgSP #2) and ARPE-19/pT2.CAn.hoG #3 (ppG #3) showing correct processing of GDNF. Samples were electrophoresed on 15% SDS gels and subsequently transferred to PVDF membranes. The blocked membrane was incubated with anti-GDNF antibody (R&D Systems, AF-212-NA) followed by HRP-linked anti-goat anti-body and detection with ECL. Purified recombinant GDNF from R&D Systems is included as reference. This protein lacks 31 amino acid residues from the amino-terminus of the predicted sequence, leading to a slightly smaller MW (11.6 kDa predicted for the non-glycosylated monomer).

FIG. 4 describes GDNF release from devices filled with cell clones as indicated. GDNF release from 1 to 4 weeks after device filling is shown for each cell clone.

FIG. 5 describes GDNF release from devices filled with the different clones, measured before implantation (2.5 weeks after filling) and after explantation. Data are shown as the mean±SEM.

FIG. 6 describes hematoxylin stained sections of device #73 and #74 with clone ppG-120 showing good cell survival after 2 weeks in rat brain.

FIG. 7 describes hematoxylin stained sections of device #69 and #70 with clone ppG-125 showing good cell survival after 2 weeks in rat brain.

FIG. 8 describes GDNF immunohistochemistry on brain sections, covering the implant site in the striatum for rat #1-3 with clone ppG-2 in the left side and ppG-20 in the right side.

FIG. 9 describes GDNF immunohistochemistry on brain sections, covering the implant site in the striatum for rat #19-21 with clone ppG-120 and IgSP-2g placed as indicated.

FIG. 10 describes GDNF tissue levels measured around implanted devices with GDNF-producing clones. Especially clone ppG-2, ppG-20, ppG-120 and ppG-125 gave high GDNF tissue levels. Punches taken from the striatum in untreated rats were included as negative control. Note that the optical density measured for samples from one of the ppG-48 devices and all of the ppG-125 devices were outside the standard curve, and the values shown are therefore underestimated.

FIG. 11 describes a GDNF Western blot of homogenized tissue samples from selected implant sites. The device number and clone ID is indicated. The negative control (1^{st} lane) is from the striatum of an untreated rat. Purified recombinant GDNF from R&D Systems is included as reference. This protein lacks 31 amino acid residues from the amino-terminus of the predicted sequence, leading to a slightly smaller MW (11.6 kDa predicted for the non-glycosylated monomer). Monomers and dimers of glycosylated and non-glycosylated GDNF is seen (indicated by arrows); no proGDNF was detected.

FIG. 12 describes a horizontal view of the rat brain showing the placement of the device (green) and the 6-OHDA injections (yellow) in the striatum.

FIG. 13 describes GDNF release from devices in media samples collected before implantation and after explantation. Data are shown as the mean±SEM.

FIG. 14 describes hematoxylin stained sections of device #33 and #34 with clone ppG-120 showing good cell survival after explantation at the termination of the 6-OHDA experiment (devices seven weeks in rat brain). The GDNF release measured from the shown devices after explantation is shown in blue.

FIG. 15 describes hematoxylin stained sections of device #53 and #55 with clone ppG-125 showing good cell survival after explantation at the termination of the 6-OHDA experiment (devices seven weeks in rat brain). The GDNF release measured from the shown devices after explantation is shown in blue.

FIG. 16 describes GDNF immunohistochemistry on sections with implant sites (i) for device #53 and #55 (clone ppG-125) in rat 28 and 29. The GDNF-immunoreactivity (brown color) illustrates that the GDNF protein secreted from the implanted devices diffuses well and covers all the striatum (Str).

FIG. 17 describes FIG.s from Paxinos rat brain atlas (1997) showing the sections chosen for evaluation of the striatal 6-OHDA lesion.

FIG. 18 describes image analyses of the individual rats in the control group with empty devices (A) Quantification of tyrosine hydroyxlase immunoreactivity in the lesion side as % of the control side in four sections from striatum (Bregma 1.0, 0.2, −0.4 and −1.0). The dotted lines indicate 50 and 100% of the control side, respectively. Animals with a mean striatal tyrosine hydroyxlase immunoreactivity of less than 50% are shown with blue arrows. (B) Quantification of tyrosine hydroyxlase immunoreactivity in the lesion side as % of the control side in three sections from substantia nigra (Bregma −4.8, −5.2, −5.6). The dotted line indicates 100% of the control side. Animals should have a sufficient striatal 6-OHDA lesion (less than 50% tyrosine hydroyxlase immunoreactivity compared with control side) to be included in the final analyses (green arrows).

FIG. 19 describes image analyses of the individual rats in the group with ppG-120 devices (A) Quantification of tyrosine hydroyxlase immunoreactivity in the lesion side as of the control side in four sections from striatum (Bregma 1.0, 0.2, −0.4 and −1.0). The dotted lines indicate 50 and 100% of the control side, respectively. Animals with a mean striatal tyrosine hydroyxlase immunoreactivity of less than 50% are shown with blue arrows. (B) Quantification of tyrosine hydroyxlase immunoreactivity in the lesion side as % of the control side in three sections from substantia nigra (Bregma −4.8, −5.2, −5.6). The dotted line indicates 100% of the control side. Animals should have a sufficient striatal 6-OHDA lesion (less than 50% tyrosine hydroyxlase immunoreactivity compared with control side) to be included in the final analyses (green arrows).

FIG. 20 describes image analyses of the individual rats in the group with ppG-125 devices. (A) Quantification of tyrosine hydroyxlase immunoreactivity in the lesion side as % of the control side in four sections from striatum (Bregma 1.0, 0.2, −0.4 and −1.0). The dotted lines indicate 50 and 100% of the control side, respectively. Animals with a mean striatal tyrosine hydroyxlase immunoreactivity of less than 50% are shown with blue arrows. (B) Quantification of tyrosine hydroyxlase immunoreactivity in the lesion side as % of the control side in three sections from substantia nigra (Bregma −4.8, −5.2, −5.6). The dotted line indicates 100% of the control side. Animals should have a sufficient striatal 6-OHDA lesion (less than 50% TH immunoreactivity compared with control side) to be included in the final analyses (green arrows).

FIG. 21 describes results of image analyses showing neuroprotective effect of devices with ppG-120 and ppG-125 on DA neurons in substantia nigra (SN). Data in the columns show the mean percentage ±SEM of surviving tyrosine hydroyxlase-positive neurons (corresponding to the tyrosine hydroyxlase-immunoreactive area) in substantia nigra in the lesion side. In addition, the mean values for the individual rats are shown by a vertical point plot. Group with empty devices (n=4), group with ppG-120 (n=8) and group with ppG-125 (n=7).

FIG. 22 describes results of manual cell countings in substantia nigra (SN) showing neuroprotective effect of devices with ppG-120 and ppG-125 on the DA neurons. Data in the columns shows the mean percentage ±SEM of surviving tyrosine hydroyxlase-positive neurons in substantia nigra in the lesion side. In addition, the mean values for the individual rats are shown by a vertical point plot. (A) Results with animals having small lesion excluded: Group with empty devices (n=4), group with ppG-120 (n=8) and group with ppG-125 (n=7). (B) Results with all animals included.

FIG. 23 describes tyrosine hydroyxlase immunostaining of substantia nigra control side and lesion side in a rat from the ppG-125 group (#29). In the lesion side, many of the surviving neurons show down regulation of tyrosine hydroyxlase expression (indicated by blue arrows), compared with the normal expression level (red arrows).

FIG. 24 is a panoramic view of a section through the middle and inner ear of a test subject (12 weeks, left side, level 10). Arrows indicate the area of device (which was removed prior to sectioning) and surrounding, localized fibrosis and inflammation.

FIG. 25 is a higher magnification view of sheath around an implant tract (indicated by arrows) which is filled with blood for a test subject (12 weeks, left side, level 8). A circumferential ring of fibrous connective tissue and eccentric chronic inflammation can be seen surrounding the transplant site.

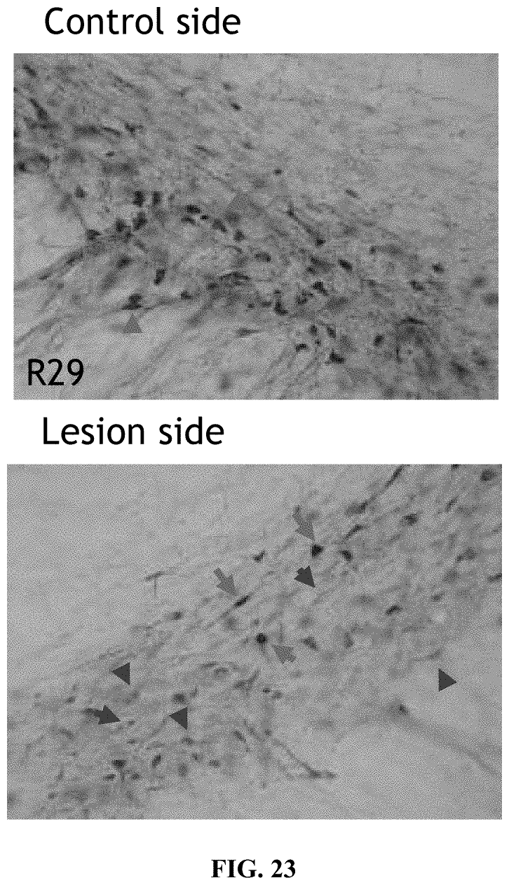

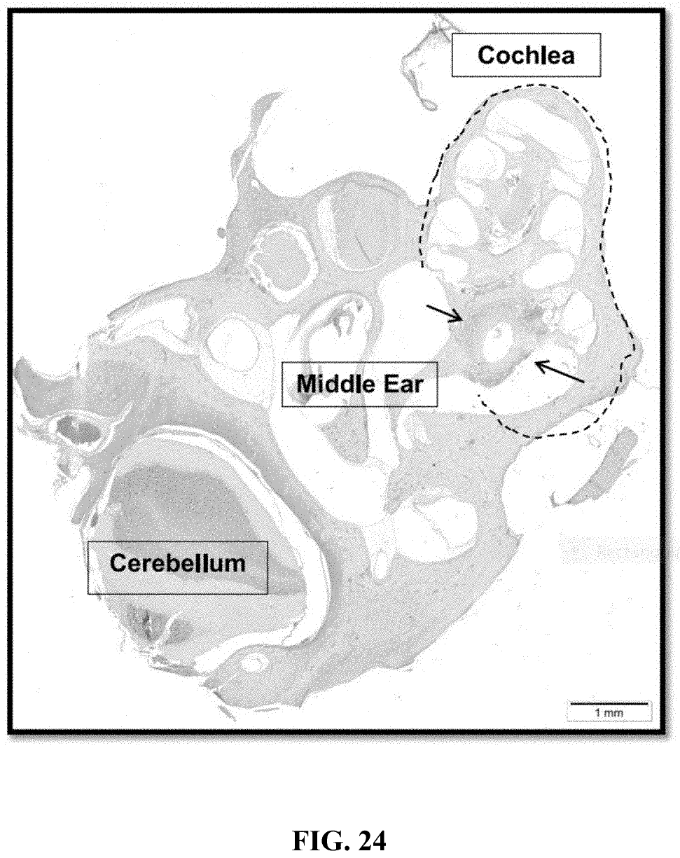

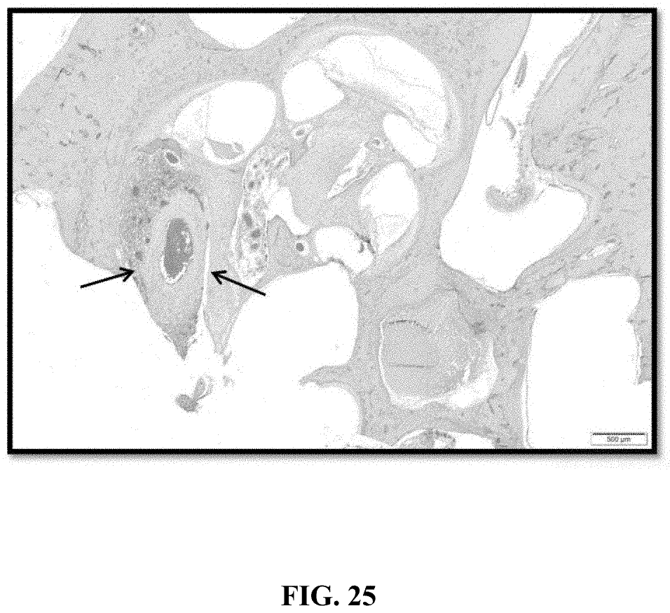

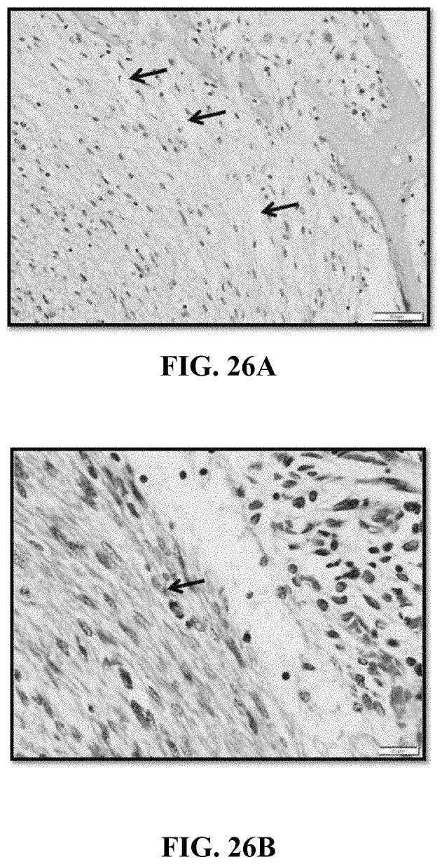

FIGS. 23A and 23B are a high magnification images of a test subject (12 weeks, left side) evidencing swelling of myelin sheaths (indicated by arrows) in the nerve from indicating minimal nerve injury (H&E, 20×) (FIGS. 23A) and focal apoptotic debris (indicated by an arrow) in the nerve indicating minimal nerve injury (H&E, 20×) (FIG. 23B).

### Definitions

A signal peptide or a eukaryotic signal peptide, as used herein, is a peptide present on proteins that are destined to either be secreted or to be membrane components. It is usually N-terminal to the protein. In the present context, all signal peptides identified in SignalP (version 2.0 or preferably version 3.0) as signal peptides are considered a signal peptide.

A mammalian signal peptide, as used herein, is a signal peptide derived from a mammalian protein secreted through the endoplasmic reticulum.

A heterologous signal peptide, as used herein, is a signal peptide not naturally being operatively linked to a GDNF polypeptide.

Mature human GDNF polypeptide, as used herein, means the 134 amino acids of native human GDNF, i.e., amino acids 1-134 of SEQ ID No. 1, and processed into a dimer. In certain contexts, it will be understood that “secreted GDNF polypeptide” means a polypeptide to be secreted as opposed to one that has already been secreted.

Sequence identity, as used herein, refers to identity between a reference amino acid sequence and a variant amino acid sequences is performed by aligning the sequences using the default settings of Clustal W (1.82). The number of fully conserved residues is counted and divided by the number of residues in the reference sequence.

### DETAILED DESCRIPTION OF THE INVENTION

### I. Signal Sequences

The targeting of secreted and proteins to the secretory pathway is accomplished via the attachment of a short, amino-terminal sequence, known as the signal peptide or signal sequence (von Heijne, (1985), J Mol Biol, 184:99-105; Kaiser and Botstein, (1986), Mol Cell Biol, 6:2382-91). The signal peptide itself contains several elements necessary for optimal function, the most important of which is a hydrophobic component. Immediately preceding the hydrophobic sequence is often a basic amino acid or acids, whereas at the carboxyl-terminal end of the signal peptide are a pair of small, uncharged amino acids separated by a single intervening amino acid which defines the signal peptidase cleavage site.

A preferred mammalian signal peptide is from 15 to 30 amino acids long (average for eukaryotes is 23 amino acids). The common structure of signal peptides from various proteins is commonly described as a positively charged n-region, followed by a hydrophobic h-region and a neutral but polar c-region. The (−3,−1)-rule states that the residues at positions −3 and −1 (relative to the cleavage site) must be small and neutral for cleavage to occur correctly.

The n-region of eukaryotic signal sequences is only slightly Arginine rich. The h-region is short and very hydrophobic. The c-region is short and has no observable pattern. As described the −3 and −1 positions consist of small and neutral residues. The amino acid residues C-terminal to the cleavage site is of less importance in eukaryotes.

In the C-region the residues at position −1 and −3 are the most important. These are small, uncharged amino acids. At position −1 the residue is preferably A, G, S, I, T or C. More preferably the −1 position is A, G or S. At position −3 the residue is preferably A, V, S, T, G, C, I, or D. More preferably, the −3 position is A, V, S or T.

The hydrophobic region prevalently consist of a hydrophobic residues. These include A, I, L, F, V, and M. Preferably, at positions −6 to −13. Of the 8 amino acids constituting this region, at least 4 residues should be hydrophobic, more preferably at least 5, more preferably at least 6, such as 7 or 8.

Various different signal peptides can be used in the GDNF constructs according to the present invention. The signal peptide can be any functional signal peptide, such as a heterologous signal peptide such as an Immunoglobulin Signal Peptide (IgSP). The signal peptide may be from any suitable species such as human, mouse, rat, monkey, pig. Preferably it is from human.

As evidenced by the appended examples, the use of the IgSP without the GDNF pro-peptide in general results in an improved secretion of bioactive GDNF both in vitro and in vivo. The results were reproducible with plasmid transfected cells. The cells secrete the mature protein as a biologically active protein, when the IgSP coding sequence is fused directly to the gene coding for the mature protein, excluding the native pre-pro part of GDNF (SEQ ID No. 2). In an embodiment, the encoded signal peptide is a mouse Ig heavy chain gene V-region.

As evidenced by the appended examples the use of this signal peptides in general results in an improved secretion of the encoded GDNF. The results were reproducible with plasmid transfected cells. The cells produce the mature protein in the correct size even when the IgSP gene is fused directly to the gene coding for the mature protein (i.e., excluding the prepro part).

Preferably the IgSP is of murine or human origin because the murine IgSP is known to be functional in mouse, rat and human beings. For use in human beings, the IgSP preferably is of human origin in order to reduce the risk of any cross species side effect.

The nucleotide sequence coding for human GDNF prepropeptide is set forth in SEQ ID NO: 3 of the present application. The encoded protein is 211 amino acids long and is set forth in SEQ ID NO: 4. Preferably the GDNF used in the context of the present invention is human mature GDNF, but it is likewise contemplated that the corresponding mouse and rat sequences can be used.

Sequence variants of the present invention are suitably defined with reference to the encoded biologically active GDNF. It is contemplated that the sequence of GDNF can be changed without changing the biological activity of the growth factor. In one embodiment of the present invention a sequence variant of GDNF is a sequence encoding a growth factor, which shares at least 70% sequence identity to the amino acids of human or mouse or rat GDNF (SEQ ID Nos. 1, 5 and 6). More preferably the sequence variant shares at least 75% sequence identity to said GDNF, more preferably at least 80%, more preferably at least 85%, more preferably at least 90%, more preferably at least 95%, more preferably at least 97%, more preferably at least 99%.

Mutations can be introduced into GDNF, by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the GDNF protein is replaced with another amino acid residue from the same side chain family.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, VLIM, HFY, wherein the letters within each group represent the single letter amino acid code.

### II. Target Tissues for Treatment of Neurodegenerative Disorders

One important parameter for in vivo gene therapy is the selection of a suitable target tissue. A region of the brain is selected for its retained responsiveness to neurotrophic factors in particular to GDNF. In humans, CNS neurons, which retain responsiveness to neurotrophic factors into adulthood include the cholinergic basal forebrain neurons, the entorhinal cortical neurons, the thalamic neurons, the locus coeruleus neurons, the spinal sensory neurons and the spinal motor neurons. A further characteristic of cells with retained responsiveness to GDNF is the expression of Ret and one of the two co-receptors GFRα1 and GFRα2.

Abnormalities within the cholinergic compartment of this complex network of neurons have been implicated in a number of neurodegenerative disorders, including AD, Parkinson's disease, and amyotrophic lateral sclerosis (ALS, also known as Lou Gehrig's disease). The cholinergic basal forebrain (particularly, the Ch4 region of the basal forebrain) is a particularly suitable target tissue.

Within the primate forebrain, magnocellular neurons Ch1-Ch4 provide cholinergic innervation to the cerebral cortex, thalamus and basolateral nucleus of the amygdala. In subjects with neurodegenerative diseases such as AD, neurons in the Ch4 region (nucleus basalis of Meynert) which have nerve growth factor (NGF) receptors undergo marked atrophy as compared to normal controls (See, e.g., Kobayashi et al., (1991), Mol Chem Neuropathol, 15:193-206). In normal subjects, neurotrophins prevent sympathetic and sensory neuronal death during development and prevents cholinergic neuronal degeneration in adult rats and primates (Tuszynski et al., (1996), Gene Thera, 3:305314). The resulting loss of functioning neurons in this region of the basal forebrain is believed to be causatively linked to the cognitive decline experienced by subjects suffering from neurodegenerative conditions such as AD (Tuszynski et al., supra and, Lehericy et al., (1993), J Comp Neurol, 330:15-31).

### III. Dosing Requirements and Delivery Protocol

Guidance as to the dosing of GDNF in the treatment of Parkinson's Disease can be found in the numerous cited references concerning delivery of GDNF using in vivo gene therapy.

In a preferred embodiment, the cells excreting GDNF are introduced into the target area by an implantable capsule, as described in U.S. Pat. Nos. 9,364,427 and 9,669,154, both of which are incorporated by reference herein.

In a preferred embodiment, the administration site is the striatum of the brain, in particular the caudate and/or the putamen. Insertion of the cells of the invention into the putamen can label target sites located in various distant regions of the brain, for example, the globus pallidus, amygdala, subthalamic nucleus or the substantia nigra. Transduction of cells in the pallidus commonly causes retrograde labelling of cells in the thalamus. In a preferred embodiment the (or one of the) target site(s) is the substantia nigra. Insertion may also be into both the striatum and the substantia nigra.

### IV. Expression Vectors

Construction of vectors for recombinant expression of GDNF for use in the invention may be accomplished using conventional techniques which do not require detailed explanation to one of ordinary skill in the art. For review, however, those of ordinary skill may wish to consult Maniatis et al., in Molecular Cloning: A Laboratory Manual, Cold Spring Harbor Laboratory, (NY 1982).

The chimeric expression constructs used in the present invention may be created as described in the examples, e.g., by amplifying the desired fragments (a signal sequence and a GDNF coding sequence) by PCR and fusing these in overlapping PCR. As several of the preferred signal sequences are relatively short, the 5′ PCR primer used for amplifying the GDNF coding sequence may include the sequence coding for the signal sequence as well as a TATA box and other regulatory elements.

Briefly, construction of recombinant expression vectors employs standard ligation techniques. For analysis to confirm correct sequences in vectors constructed, the genes are sequences using, for example, the method of Messing et al., ((1981), Nucleic Acids Res, 9(2):309-21), the method of Maxam and Gilbert, (1980), Methods Enzymol, 65(1):499-560), or other suitable methods which will be known to those skilled in the art.

Size separation of cleaved fragments is performed using conventional gel electrophoresis as described, for example, by Maniatis et al., ((1982), Molecular Cloning, pp. 133-4).

Expression of a gene is controlled at the transcription, translation or post-translation levels. Transcription initiation is an early and critical event in gene expression. This depends on the promoter and enhancer sequences and is influenced by specific cellular factors that interact with these sequences. The transcriptional unit of many genes consists of the promoter and in some cases enhancer or regulator elements (Banerji et al., (1981), Cell 27:299); Corden et al., (1980), Science, 209:1406); and Breathnach and Chambon, (1981), Ann Rev Biochem, 50:349). For retroviruses, control elements involved in the replication of the retroviral genome reside in the long terminal repeat (LTR) (Weiss et al., eds., The Molecular Biology of Tumor Viruses: RNA Tumor Viruses, Cold Spring Harbor Laboratory, (NY 1982)). Moloney murine leukemia virus (MLV) and Rous sarcoma virus (RSV) LTRs contain promoter and enhancer sequences (Jolly et al., (1983), Nucleic Acids Res, 11:1855; Capecchi et al., In: Enhancer and Eukaryotic Gene Expression, Gulzman and Shenk, eds., pp. 101-2, Cold Spring Harbor Laboratories (NY 1991). Other potent promoters include those derived from cytomegalovirus (CMV) and other wild-type viral promoters.

Promoter and enhancer regions of a number of non-viral promoters have also been described (Schmidt et al., (1985), Nature, 314:285; Rossi and deCrombrugghe, (1987), Proc Natl Acad Sci USA, 84:5590-4). Methods for maintaining and increasing expression of transgenes in quiescent cells include the use of promoters including collagen type I (1 and 2) (Prockop and Kivirikko, (1984), N Eng J Med, 311:376); Smith and Niles, (1980), Biochemistry, 19:1820; de Wet et al., (1983), J Biol Chem, 258:14385), SV40 and LTR promoters.

According to one embodiment of the invention, the promoter is a constitutive promoter selected from the group consisting of: ubiquitin promoter, CMV promoter, JeT promoter (U.S. Pat. No. 6,555,674), SV40 promoter, and Elongation Factor 1 alpha promoter (EF1-alpha).

Examples of inducible/repressible promoters include: Tet-On, Tet-Off, Rapamycin-inducible promoter, M×1.

In addition to using viral and non-viral promoters to drive transgene expression, an enhancer sequence may be used to increase the level of transgene expression. Enhancers can increase the transcriptional activity not only of their native gene but also of some foreign genes (Armelor, (1973), Proc Natl Acad Sci USA, 70:2702). For example, in the present invention collagen enhancer sequences may be used with the collagen promoter 2 (I) to increase transgene expression. In addition, the enhancer element found in SV40 viruses may be used to increase transgene expression. This enhancer sequence consists of a 72 base pair repeat as described by Gruss et al., (1981), Proc Natl Acad Sci USA, 78:943; Benoist and Chambon, (1981), Nature. 290:304, and Fromm and Berg, (1982), J Mol Appl Genetics, 1:457, all of which are incorporated by reference herein. This repeat sequence can increase the transcription of many different viral and cellular genes when it is present in series with various promoters (Moreau et al., (1981), Nucl Acids Res, 9:6047.

Further expression enhancing sequences include but are not limited to Woodchuck hepatitis virus post-transcriptional regulation element, WPRE, SP163, rat Insulinll-intron or other introns, CMV enhancer, and Chicken [beta]-globin insulator or other insulators.

Transgene expression may also be increased for long term stable expression using cytokines to modulate promoter activity. Several cytokines have been reported to modulate the expression of transgene from collagen 2 (I) and LTR promoters (Chua et al., (1990), Connective Tiss Res., 25:161-170; Elias et al., (1990), Annals NY Acad Sci, 580:233-44); Seliger et al., (1988), J Immunol, 141: 2138-44 and Seliger et al., (1988), J Virol. 62:619-21). For example, transforming growth factor (TGF), interleukin (IL)-I, and interferon (INF) down regulate the expression of transgenes driven by various promoters such as LTR. Tumor necrosis factor (TNF) and TGF 1 up regulate, and may be used to control, expression of transgenes driven by a promoter. Other cytokines that may prove useful include basic fibroblast growth factor (bFGF) and epidermal growth factor (EGF).

Collagen promoter with the collagen enhancer sequence (Coll (E)) may also be used to increase transgene expression by suppressing further any immune response to the vector which may be generated in a treated brain notwithstanding its immune-protected status. In addition, anti-inflammatory agents including steroids, for example dexamethasone, may be administered to the treated host immediately after vector composition delivery and continued, preferably, until any cytokine-mediated inflammatory response subsides. An immunosuppression agent such as cyclosporin may also be administered to reduce the production of interferons, which downregulates LTR promoter and Coll (E) promoter-enhancer and reduces transgene expression.

The vector may comprise further sequences such as a sequence coding for the Cre-recombinase protein, and LoxP sequences. A further way of ensuring temporary expression of the neublastin is through the use of the Cre-LoxP system which results in the excision of part of the inserted DNA sequence either upon administration of Cre-recombinase to the cells (Daewoong et al., Nat Biotechnol, 19:929-33) or by incorporating a gene coding for the recombinase into the virus construct (Pluck, (1996), Int J Exp Path, 77:269-78). Incorporating a gene for the recombinase in the virus construct together with the LoxP sites and a structural gene (a neublastin in the present case) often results in expression of the structural gene for a period of approximately five days.

### V. Pharmaceutical Preparations

To form a GDNF composition for use in the invention, GDNF encoding expression vectors may be placed into a pharmaceutically acceptable suspension, solution or emulsion. Suitable mediums include saline and liposomal preparations.

More specifically, pharmaceutically acceptable carriers may include sterile aqueous of nonaqueous solutions, suspensions, and emulsions. Examples of nonaqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils.

Intravenous vehicles include fluid and nutrient replenishers, electrolyte replenishers (such as those based on Ringer's dextrose), and the like.

Preservatives and other additives may also be present such as, for example, antimicrobials, antioxidants, chelating agents, and inert gases and the like. Further, a composition of GDNF transgenes may be lyophilized using means well known in the art, for subsequent reconstitution and use according to the invention.

A colloidal dispersion system may also be used for targeted gene delivery.

Colloidal dispersion systems include macromolecule complexes, nanocapsules, microspheres, beads, and lipid-based systems including oil-in-water emulsions, micelles, mixed micelles, and liposoms. Liposomes are artificial membrane vesicles which are useful as delivery vehicles in vitro and in vivo. It has been shown that large unilamellar vesicles (LUV), which range in size from 0.2-4.0 μm can encapsulate a substantial percentage of an aqueous buffer containing large macro molecules. RNA, DNA and intact virions can be encapsulated within the aqueous interior and be delivered to cells in a biologically active form (Fraley et al., (1981), Trends Biochem Sci, 6:77). In addition to mammalian cells, liposomes have been used for delivery of operatively encoding transgenes in plant, yeast and bacterial cells. In order for a liposome to be an efficient gene transfer vehicle, the following characteristics should be present: (1) encapsulation of the genes encoding the GDNF at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Mannino et al., (1988), Biotechniques, 6:682).

The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine and di stearoylphosphatidylcholine.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs which contain sinusoidal capillaries.

Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted gene delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand.

A further example of a delivery system includes transplantation into the therapeutic area of a composition of packaging cells capable of producing vector particles as described in the present invention. Methods for encapsulation and transplantation of such cells are known in the art, in particular from WO 97/44065 (Cytotherapeutics). By selecting a packaging cell line capable of producing lentiviral particles, transduction of non-dividing cells in the therapeutic area is obtained. By using retroviral particles capable of transducing only dividing cells, transduction is restricted to de-novo differentiated cells in the therapeutic area.

### VI. Encapsulation of Cells

Encapsulated cell therapy is based on the concept of isolating cells from the recipient host's immune system by surrounding the cells with a semipermeable biocompatible material before implantation within the host. The invention includes a device in which GDNF—secreting cells are encapsulated in an immunoisolatory capsule. An “immunoisolatory capsule” means that the capsule, upon implantation into a recipient host, minimizes the deleterious effects of the host's immune system on the cells in the core of the device. Cells are immunoisolated from the host by enclosing them within implantable polymeric capsules formed by a microporous membrane. This approach prevents the cell-to cell contact between host and implanted tissues, eliminating antigen recognition through direct presentation. The membranes used can also be tailored to control the diffusion of molecules, such as antibody and complement, based on their molecular weight (Lysaght et al., (1994), J Cell Biochem, 56:196-204, Colton, (1996), Trends Biotechnol, 14:158-62). Using encapsulation techniques, cells can be transplanted into a host without immune rejection either with or without use of immunosuppressive drugs. Useful biocompatible polymer capsules usually contain a core that contains cells, either suspended in a liquid medium or immobilized within an immobilizing matrix, and a surrounding or peripheral region of permselective matrix or membrane (“jacket”) that does not contain isolated cells, that is biocompatible, and that is sufficient to protect cells in the core from detrimental immunological attack. Encapsulation hinders elements of the immune system from entering the capsule, thereby protecting the encapsulated cells from immune destruction. The semipermeable nature of the capsule membrane also permits the biologically active molecule of interest to easily diffuse from the capsule into the surrounding host tissue.

The capsule can be made from a biocompatible material. A “biocompatible material” is a material that, after implantation in a host, does not elicit a detrimental host response sufficient to result in the rejection of the capsule or to render it inoperable, for example through degradation. The biocompatible material is relatively impermeable to large molecules, such as components of the host's immune system, but is permeable to small molecules, such as insulin, growth factors, and nutrients, while allowing metabolic waste to be removed. A variety of biocompatible materials are suitable for delivery of growth factors by the composition of the invention. Numerous biocompatible materials are known, having various outer surface morphologies and other mechanical and structural characteristics. Preferably, the capsule of this invention will be similar to those described by U.S. Pat. Nos. 9,364,427 and 9,669,154, both of which are incorporated by reference. Such capsules allow for the passage of metabolites, nutrients and therapeutic substances while minimizing the detrimental effects of the host immune system. Components of the biocompatible material may include a surrounding semipermeable membrane and the internal cell-supporting scaffolding. Preferably, the transformed cells are seeded onto the scaffolding, which is encapsulated by the permselective membrane. The filamentous cell-supporting scaffold may be made from any biocompatible material selected from the group consisting of acrylic, polyester, polyethylene, polypropylene polyacetonitrile, polyethylene teraphthalate, nylon, polyamides, polyurethanes, polybutester, silk, cotton, chitin, carbon, or biocompatible metals. Also, bonded fiber structures can be used for cell implantation (U.S. Pat. No. 5,512,600, incorporated by reference herein). Biodegradable polymers include those comprised of poly(lactic acid) PLA, poly(lactic-coglycolic acid) PLGA, and poly(glycolic acid) PGA and their equivalents. Foam scaffolds have been used to provide surfaces onto which transplanted cells may adhere (WO 98/05304, incorporated by reference herein). Woven mesh tubes have been used as vascular grafts (WO 99/52573, incorporated by reference herein). Additionally, the core can be composed of an immobilizing matrix formed from a hydrogel, which stabilizes the position of the cells. A hydrogel is a 3-dimensional network of cross-linked hydrophilic polymers in the form of a gel, substantially composed of water.

Various polymers and polymer blends can be used to manufacture the surrounding semipermeable membrane, including polyacrylates (including acrylic copolymers), polyvinylidenes, polyvinyl chloride copolymers, polyurethanes, polystyrenes, polyamides, cellulose acetates, cellulose nitrates, polysulfones (including polyether sulfones), polyphosphazenes, polyacrylonitriles, poly(acrylonitrile/covinyl chloride), as well as derivatives, copolymers and mixtures thereof. Preferably, the surrounding semipermeable membrane is a biocompatible semipermeable hollow fiber membrane. Such membranes, and methods of making them are disclosed by U.S. Pat. Nos. 5,284,761 and 5,158,881, both of which are incorporated by reference herein. The surrounding semipermeable membrane is formed from a polyether sulfone hollow fiber, such as those described by U.S. Pat. Nos. 4,976,859 or 4,968,733, both of which are incorporated by reference herein. An alternate surrounding semipermeable membrane material is poly(acrylonitrile/covinyl chloride).

The capsule can be any configuration appropriate for maintaining biological activity and providing access for delivery of the product or function, including for example, cylindrical, rectangular, disk-shaped, patch-shaped, ovoid, stellate, or spherical. Moreover, the capsule can be coiled or wrapped into a mesh-like or nested structure. If the capsule is to be retrieved after it is implanted, configurations which tend to lead to migration of the capsules from the site of implantation, such as spherical capsules small enough to travel in the recipient host's blood vessels, are not preferred. Certain shapes, such as rectangles, patches, disks, cylinders, and flat sheets offer greater structural integrity and are preferable where retrieval is desired.

When macrocapsules are used, preferably between 10³ and 10⁸ cells are encapsulated, most preferably 10⁵ to 10⁷ cells are encapsulated in each device. Dosage may be controlled by implanting a fewer or greater number of capsules, preferably between 1 and 10 capsules per patient.

The scaffolding may be coated with extracellular matrix (ECM) molecules. Suitable examples of extracellular matrix molecules include, for example, collagen, laminin, and fibronectin. The surface of the scaffolding may also be modified by treating with plasma irradiation to impart charge to enhance adhesion of cells.

Any suitable method of sealing the capsules may be used, including the use of polymer adhesives or crimping, knotting and heat sealing. In addition, any suitable “dry” sealing method can also be used, as described, e.g., in U.S. Pat. No. 5,653,687, incorporated by reference herein.

The encapsulated cell devices are implanted according to known techniques. Many implantation sites are contemplated for the devices and methods of this invention. These implantation sites include, but are not limited to, the central nervous system, including the brain, spinal cord (See, U.S. Pat. Nos. 5,106,627, 5,156,844 and 5,554,148, all of which are incorporated by reference herein), and the aqueous and vitreous humors of the eye (WO 97/34586, incorporated by reference herein).

The ARPE-19 cell line is a superior platform cell line for encapsulated cell based delivery technology and is also useful for unencapsulated cell based delivery technology. The ARPE-19 cell line is hardy (i.e., the cell line is viable under stringent conditions, such as implantation in the central nervous system or the intra-ocular environment). ARPE-19 cells can be genetically modified to secrete a substance of therapeutic interest. ARPE-19 cells have a relatively long life span. ARPE-19 cells are of human origin. Furthermore, encapsulated ARPE-19 cells have good in vivo device viability. ARPE-19 cells can deliver an efficacious quantity of growth factor. ARPE-19 cells elicit a negligible host immune reaction. Moreover, ARPE-19 cells are non-tumorigenic.

Methods and apparatus for implantation of capsules into the CNS are described in U.S. Pat. No. 5,487,739, incorporated by reference herein.

In one aspect the invention relates to a biocompatible capsule comprising: a core comprising living packaging cells that secrete a viral vector for infection of a target cell, wherein the viral vector is a vector according to the invention; and an external jacket surrounding said core, said jacket comprising a permeable biocompatible material, said material having a porosity selected to permit passage of retroviral vectors of approximately 100 nm diameter thereacross, permitting release of said viral vector from said capsule.

Preferably, the core additionally comprises a matrix, the packaging cells being immobilized by the matrix. According to one embodiment, the jacket comprises a hydrogel or thermoplastic material.

Methods and devices for encapsulation of packaging cells are disclosed in U.S. Pat. No. 6,027,721, incorporated by reference herein.

### VII. Medical Use and Methods of Treatment

In one aspect the invention relates to the use of the vector according to the invention for the preparation of a medicament for the treatment of a nervous system disorder. The nervous system disorder can be a disorder of the peripheral nervous system or the central nervous system.

By treatment is not only intended curative treatment but also preventive (not absolute prevention) or prophylactic treatment. Treatment may also be ameliorative or symptomatic.

Preferably the CNS disorder is a neurodegenerative or neurological disease. The neurodegenerative or neurological disease may be a disease involving lesioned and traumatic neurons, such as traumatic lesions of peripheral nerves, the medulla, the spinal cord, cerebral ischemic neuronal damage, neuropathy, peripheral neuropathy, neuropathic pain, Alzheimer's disease, Huntington's disease, Parkinson's disease, amyotrophic lateral sclerosis, memory impairment connected to dementia. The neurodegenerative component of multiple sclerosis is also treatable according to the present invention.

According to one preferred embodiment of the invention, the neurodegenerative disease is Parkinson's disease (See the examples).

In another preferred embodiment, the disease is Amyotrophic Lateral Sclerosis or spinal cord injury.

The vectors of the present invention can also be used for the treatment of eye diseases, such as retinitis pigmentosa, macular degeneration, glaucoma, diabetic retinopathy.

Nervous system diseases may be treated by administering to an individual in need thereof a therapeutically effective amount of the invention; or a therapeutically effective amount of the pharmaceutical composition of the invention.

For Parkinson's disease, the delivery of capsules and vector is described above under “Dosing requirements and delivery protocol.” For ALS and spinal cord injury, capsules with GDNF secreting cells or virus vector may be delivered to the intrathecal space, intraventricularly or intralumbarly. For spinal cord injury, delivery may also be to the area with lesioned and/or traumatic neurons. Delivery of capsules or vector may be to the cervical/lumbar enlargement in proximity to the lower motor neurons. In particular for ALS, modified rabies virus coding for an expression construct of the present invention may be injected into afflicted muscle tissue, whereby retrograde transport to the affected motor neurons is accomplished.

Although the present invention focuses on in vivo gene therapy, it is also contemplated that, nervous system diseases can be treated by transplanting to an individual in need thereof:
i. a therapeutically effective amount of the transduced cells according to the invention;
ii. an implantable device comprising transduced cells; or
iii. a biocompatible device comprising a packaging cell line.

Said transplantation may comprise an autologous transplant, an allogeneic transplant or a xenogeneic transplant.

Most, if not all, ophthalmic diseases and disorders are associated with one or more of three types of indications: (1) angiogenesis, (2) inflammation, and (3) degeneration. To treat these disorders, the virus vectors, therapeutic cells and encapsulated cells of the present invention permit delivery of GDNF to the eye.

Delivery of vector according to the present invention may be done using subretinal injections, intravitreal injection, or transcleral injection.

Diabetic retinopathy, for example, is characterized by angiogenesis and retinal degeneration. This invention contemplates treating diabetic retinopathy by implanting devices delivering GDNF either intraocularly, preferably in the vitreous, or periocularly, preferably in the sub-Tenon's region. We most prefer delivery of capsules, naked cells, or virus vector into the vitreous for this indication. Retinopathy includes, but is not limited to, diabetic retinopathy, proliferative vitreoretinopathy, and toxic retinopathy.

Uveitis involves inflammation and secondary degeneration. This invention contemplates treating uveitis by intraocular, preferably vitreal or anterior chamber, implantation of capsules or naked cells secreting GDNF or by administering vector according to the invention to the vitreous.

Retinitis pigmentosa, by comparison, is characterized by primary retinal degeneration. This invention contemplates treating retinitis pigmentosa by intraocular, preferably vitreal, placement of devices or naked cells secreting GDNF or by administering vector according to the invention to the vitreous.

Age-related macular degeneration involves both angiogenesis and retinal degeneration. This invention contemplates treating this disorder by using the capsules or naked cells of the invention to deliver GDNF intraocularly, preferably to the vitreous, or by using the vector according to the invention to deliver GDNF intraocularly, preferably to the vitreous. Age-related macular degeneration includes, but is not limited to, dry age-related macular degeneration, exudative age-related macular degeneration, and myopic degeneration.

Glaucoma is characterized by increased ocular pressure and loss of retinal ganglion cells. Treatments for glaucoma contemplated in this invention include delivery of GDNF that protects retinal cells from glaucoma associated damage, delivered intraocularly, preferably intravitreally either by capsules, vector, or naked cells.

The present invention may be useful for the treatment of ocular neovascularization, a condition associated with many ocular diseases and disorders and accounting for a majority of severe visual loss. For example, we contemplate treatment of retinal ischemia-associated ocular neovascularization, a major cause of blindness in diabetes and many other diseases; corneal neovascularization, which predisposes patients to corneal graft failure; and neovascularization associated with diabetic retinopathy, central retinal vein occlusion, and possibly age-related macular degeneration.

In one embodiment of the present invention, living cells secreting bioactive GDNF are encapsulated and surgically inserted (under retrobulbar anesthesia) into the vitreous of the eye. For vitreal placement, the device may be implanted through the sclera, with a portion of the device or tether protruding through the sclera. Most preferably, the entire body of the device is implanted in the vitreous, with no portion of the device protruding into or through the sclera. Preferably the device is tethered to the sclera (or other suitable ocular structure). The tether may comprise a suture eyelet, or any other suitable anchoring means (See e.g., U.S. Pat. No. 6,436,427). The device can remain in the vitreous as long as necessary to achieve the desired prophylaxis or therapy. Such therapies for example include promotion of neuron or photoreceptor survival or repair, or inhibition and/or reversal of retinal or choroidal neovascularization, as well as inhibition of uveal, retinal, and optic nerve inflammation. This embodiment is preferable for delivering GDNF to the retina.

With vitreal placement, GDNF may be delivered to the retina or the RPE.

In another embodiment, cell-loaded devices are implanted periocularly, within or beneath the space known as Tenon's capsule. This embodiment is less invasive than implantation into the vitreous and thus is generally preferred. This route of administration also permits delivery of GDNF to the RPE or the retina. This embodiment is especially preferred for treating choroidal neovascularization and inflammation of the optic nerve and uveal tract. In general, delivery from this implantation site will permit circulation of GDNF to the choroidal vasculature, the retinal vasculature, and the optic nerve.

According to this embodiment we prefer periocular delivery (implanting beneath Tenon's capsule) of GDNF to the choroidal vasculature to treat macular degeneration (choroidal neovascularization).

Delivery of GDNF directly to the choroidal vasculature (periocularly) or to the vitreous (intraocularly) using the devices and methods of this invention may permit the treatment of poorly defined or occult choroidal neovascularization. It may also provide a way of reducing or preventing recurrent choroidal neovascularization via adjunctive or maintenance therapy.

Dosage can be varied by any suitable method known in the art. This includes changing (1) the number of cells per device, (2) the number of devices per eye, or (3) the level of NTN production per cell. We prefer use of 10³ to 10⁸ cells per device, more preferably 5*10⁴ to 5*10⁶ cells per device.

### VIII. Host Cells

In one aspect the invention relates to isolated host cells transduced with the vector according to the invention. These cells preferably are mammalian host cells because these are capable of secreting and processing the encoded GDNF correctly.

Preferred species include the group consisting of rodent (mouse, rat), rabbit, dog, cat, pig, monkey, human being.

Examples of primary cultures and cell lines that are good candidates for transduction with the vectors of the present invention include the group consisting of CHO, HEK293, COS, PC12, HiB5, RN33b, neuronal cells, fetal cells, ARPE-19, MDX12, C2C12, HeLa, HepG2, striatal cells, neurons, astrocytes, interneurons.

The invention also relates to cells suitable for biodelivery of GDNF via naked or encapsulated cells, which are genetically modified to overexpress GDNF, and which can be transplanted to the patient to deliver bioactive GDNF polypeptide locally. Such cells may broadly be referred to as therapeutic cells.

In a preferred embodiment of the invention, a therapeutic cell line has not been immortalized with the insertion of a heterologous immortalization gene. As the invention relates to cells which are particularly suited for cell transplantation, whether as naked cells or—preferably as encapsulated cells, such immortalized cell lines are less preferred as there is an inherent risk that they start proliferating in an uncontrolled manner inside the human body and potentially form tumors.

Preferably, the therapeutic cell line is a contact inhibited cell line. By a contact inhibited cell line is intended a cell line which when cultured in Petri dishes grow to confluency and then substantially stop dividing. This does not exclude the possibility that a limited number of cells escape the mono-layer. Contact inhibited cells may also be grown in 3D, e.g. inside a capsule. Also, inside the capsules, the cells grow to confluency and then significantly slow down proliferation rate or completely stop dividing. A particularly preferred type of cells include epithelial cells which are by their nature contact-inhibited and which form stable monolayers in culture.

Even more preferred are retinal pigment epithelial cells (RPE cells). The source of RPE cells is by primary cell isolation from the mammalian retina. Protocols for harvesting RPE cells are well-defined (Li and Turner, (1988), Exp Eye Res, 47:911-7; Lopez et al., (1989), Invest Ophthalmol Vis Sci, 30:586-8) and considered a routine methodology. In most of the published reports of RPE cell co-transplantation, cells are derived from the rat (Li and Turner, (1988); Lopez et al., (1989)). According to the present invention RPE cells are derived from humans. In addition to isolated primary RPE cells, cultured human RPE cell lines may be used in the practice of the invention.

In another embodiment the therapeutic cell line is selected from the group consisting of: human fibroblast cell lines, human astrocyte cell lines, human mesencephalic cell lines, and human endothelial cell lines, preferably immortalized with TERT, SV40T or vmyc.

The method for generating an immortalized human astrocyte cell lines has previously been described (Price et al., (1999), In Vitro Cell Dev Biol Anim, 35(5):279-88). This protocol may be used to generate astrocyte cell lines.

The following three modifications of that protocol are preferably made to generate additional human astrocyte cell lines.
Human fetal brain tissue dissected from 5-12 weeks old fetuses may be used instead of 12-16 weeks old tissue.
The immortalization gene v-myc, or TERT (telomerase) may be used instead of the SV40 T antigen.
Retroviral gene transfer may be used instead of transfection with plasmids by the calcium phosphate precipitation technique.

### IX. Support Matrix for GDNF Producing Cells

The present invention further comprises culturing GDNF producing cells in vitro on a support matrix prior to implantation into the mammalian nervous system or the eye. The pre-adhesion of cells to microcarriers prior to implantation is designed to enhance the long-term viability of the transplanted cells and provide long-term functional benefit.

To increase the long term viability of the transplanted cells, i.e., transplanted GDNF-secreting cells, the cells to be transplanted can be attached in vitro to a support matrix prior to transplantation. Materials of which the support matrix can be comprised include those materials to which cells adhere following in vitro incubation, and on which cells can grow and which can be implanted into the mammalian body without producing a toxic reaction or an inflammatory reaction which would destroy the implanted cells or otherwise interfere with their biological or therapeutic activity. Such materials may be synthetic or natural chemical substances or substances having a biological origin.

The matrix materials include, but are not limited to, glass and other silicon oxides, polystyrene, polypropylene, polyethylene, polyvinylidene fluoride, polyurethane, polyalginate, polysulphone, polyvinyl alcohol, acrylonitrile polymers, polyacrylamide, polycarbonate, polypentent, nylon, amylases, natural and modified gelatin and natural and codified collagen, natural and modified polysaccharides, including dextrans and celluloses (e.g., nitrocellulose), agar and magnetite. Either resorbable or non-resorbable materials may be used. Also intended are extracellular matrix materials, which are well-known in the art. Extracellular matrix materials may be obtained commercially or prepared by growing cells which secrete such a matrix, removing the secreting cells and allowing the cells which are to be transplanted to interact with and adhere to the matrix. The matrix material on which the cells to be implanted grow or with which the cells are mixed, may be an indigenous product of RPE cells. Thus, for example, the matrix material may be extracellular matrix or basement membrane material, which is produced and secreted by RPE cells to be implanted.

To improve cell adhesion, survival and function, the solid matrix may optionally be coated on its external surface with factors known in the art to promote cell adhesion, growth or survival. Such factors include cell adhesion molecules, extracellular matrix, such as, for example, fibronectin, laminin, collagen, elastin, glycosaminoglycans, or proteoglycans or growth factors.

Alternatively, if the solid matrix to which the implanted cells are attached is constructed of porous material, the growth- or survival promoting factor or factors may be incorporated into the matrix material, from which they would be slowly released after implantation in vivo.

When attached to the support according to the present invention, the cells used for transplantation are generally on the “outer surface” of the support. The support may be solid or porous. Even in a porous support, however, the cells are in direct contact with the external milieu without an intervening membrane or other barrier. Thus, according to the present invention, the cells are considered to be on the “outer surface” of the support even though the surface to which they adhere may be in the form of internal folds or convolutions of the porous support material which are not at the exterior of the particle or bead itself.

The configuration of the support is preferably spherical, as in a bead, but may be cylindrical, elliptical, a flat sheet or strip, a needle or pin shape, and the like. A preferred form of support matrix is a glass bead. Another preferred bead is a polystyrene bead.

Bead sizes may range from about 10 μm to 1 mm in diameter, preferably from about 90 μm to about 150 μm. For a description of various microcarrier beads, See, for example, Fisher Biotech Source 87-88, Fisher Scientific Co., (1987), pp. 72-75; Sigma Cell Culture Catalog, Sigma Chemical Co., St, Louis, (1991), pp. 162-163; Ventrex Product Catalog, Ventrex Laboratories, (1989); these references are hereby incorporated by reference herein. The upper limit of the bead's size may be dictated by the bead's stimulation of undesired host reactions, which may interfere with the function of the transplanted cells or cause damage to the surrounding tissue. The upper limit of the bead's size may also be dictated by the method of administration. Such limitations are readily determinable by one of skill in the art.

### X. In Vitro Production of GDNF

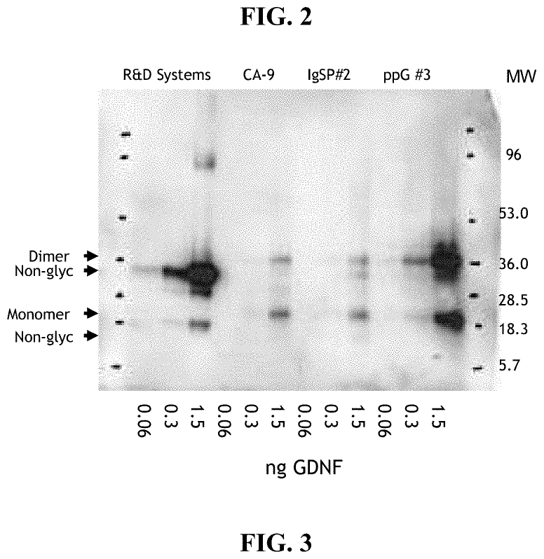

In another aspect the invention relates to a mammalian cell capable of secreting GDNF or a functional equivalent thereof in amounts in excess of 20 μg GDNF/10⁵ cells/24 hrs for more than 6 months. As shown by FIG. 2, the best plasmid transfected ARPE-19 cells produce in excess of 20 μg GDNF/10⁵ cells/24 hrs. Expression can be increased even further by the inclusion of enhancer elements such as WPRE (U.S. Pat. No. 6,136,597). Compared to the prior art BHK cells (Hoane et al., (2000), Exp Neurol. 162:189-93), these amounts are very high.

Such high producing cells may be selected from the group consisting of ARPE-19 cells, CHO cells, BHK cells, R1.1 cells, COS cells, killer cells, helper T-cells, cytotoxic T-lymphocytes and macrophages. HEK293 cells and HiB5 cells are also suitable producer cells.

GDNF or a truncated or mutated form thereof or a bioactive sequence variant can thus be produced in significant quantities by culturing these cells and recovering the GDNF from the culture medium. Mammalian produced GDNF does not need to be refolded in order to be bioactive. A further advantage is that GDNF is secreted as a mature peptide and does not include the pro-peptide. (See, FIG. 3)

These GDNF producing cells can likewise be used for therapeutic purposes and be implanted either as naked (supported or unsupported) or as encapsulated cells for localized delivery of bioactive GDNF.

### EXEMPLIFICATION

### 1. Objective

The objective of the study was to develop new cell lines with increased Glial Derived Neurotrophic Factor (GDNF) secretion for Encapsulated Cell (EC) biodelivery devices aimed at implantation for treatment of Parkinson's disease.

### 2. Summary

New expression technologies were used to develop human ARPE-19 cell derived clones with high levels of GDNF secretion. Clones were selected by in vitro and in vivo tests of GDNF secretion and survival proper-ties. Correct processing of GDNF was confirmed in vitro as well as in the tissue surrounding devices with GDNF producing clones.

From the results of the in vitro and in vivo analyses, two clones, ppG-120 and ppG-125, were selected for test of neuroprotective effect in a rat model of Parkinson's disease, the 6-hydroxy-dopamine (6-OHDA) lesion model, a collaboration with Anders Björklund's group at Lund University (Lund, Sweden).

The two clones showed significant and comparable neuroprotective effects in the rat model. As clone ppG-125 released the highest levels of GDNF before implantation, after explantation and in the tissue, it is chosen as the primary clinical cell candidate for further development.

### 3. Establishment of GDNF Expressing Cell Clones

Two technologies were employed to enhance expression of GDNF by the contact-inhibited retinal pigment epithelial cell line ARPE-19 (Dunn et al., (1996), Exp Eye Res, 62:155-69) which is well-suited for encapsulation:
1) codon-optimization of the GDNF ORF using an algorithm developed by GeneArt AG, (Regensburg, Germany); and
2) enhanced target sequence integration using the Sleeping Beauty transposon system (Ivies et al., (1997), Cell, 91:501-10).

### 3.1. Vector Development

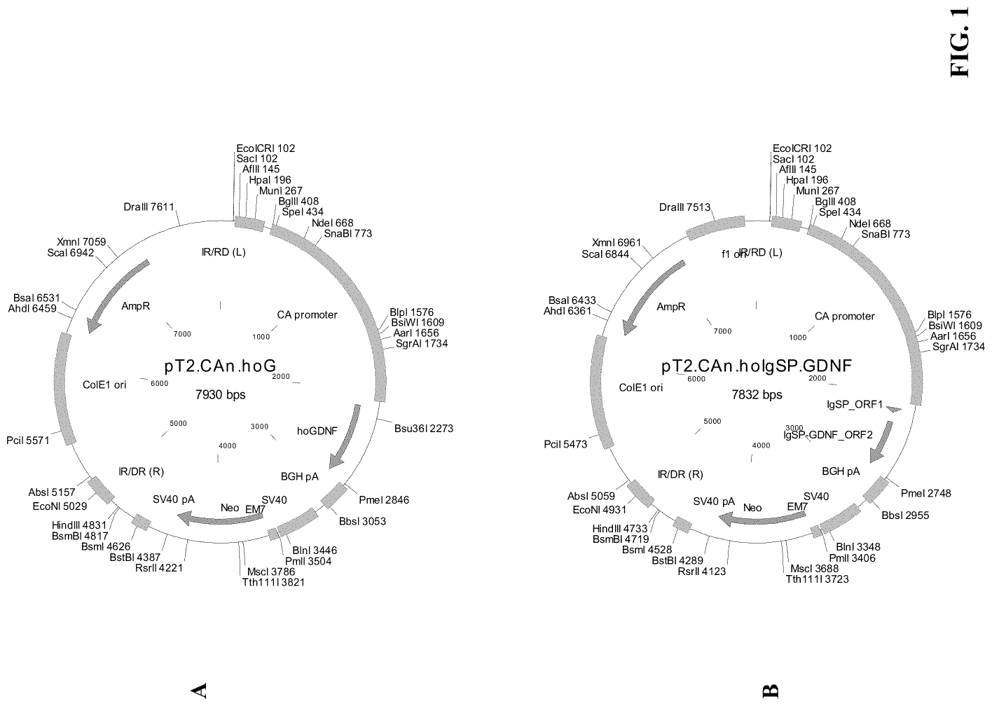

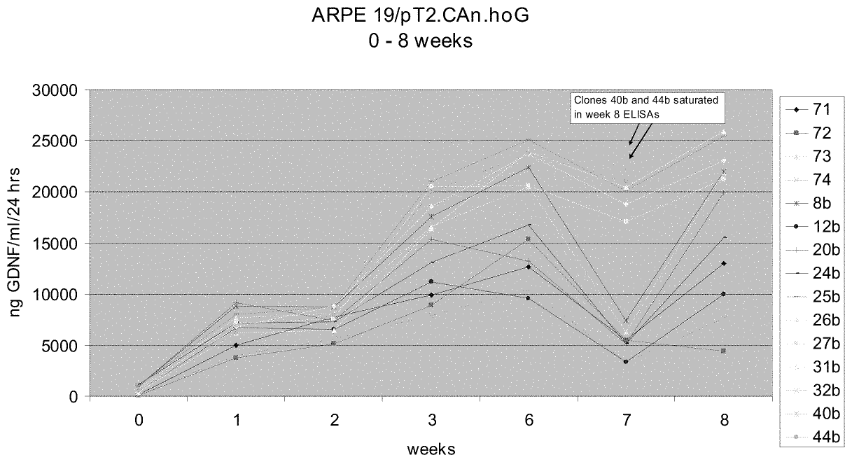

Full length human GDNF (including the prepro sequence) as well as an IgSP-GDNF chimeric version (with the prepro-region substituted by the sequence encoding the signal peptide of mouse Ig heavy chain gene V-region) were codon optimized by GeneArt AG, (Regensburg, Germany) and cloned into the pCAn expression vector under CA promoter control. Large fragments consisting of the GDNF or IgSP-GDNF and the neomycin resistance expression cassettes were excised from the pCAn construct and inserted into the vector pT2BH, an expression vector also serving as substrate vector for the Sleeping Beauty transposase (Ivies et al., (1997), Cell, 91:501-10) to create the plasmids pT2.CAn.hoG and pT2.CAn.hoIgSP.GDNF (See FIG. 1).

### 3.2. Transfection and Selection

ARPE-19, a spontaneously immortalized human RPE cell line (Dunn et al., (1996), Exp Eye Res, 62:155-69), was co-transfected with plasmids pT2.CAn.hoG and pCMV-SB-100×. The latter plasmid expresses a hyperactive version of the Sleeping Beauty transposase. The plasmid does not contain a eukaryotic selection marker cassette and is, thus, intentionally only transiently expressed. The transient expression window allows for the active, transposase-mediated integration of the Sleeping Beauty transposon, i.e., the inverted repeat Sleeping Beauty substrate sequences and the sequences contained within these repeats. Transfected cells were subsequently subjected to G418 selection and single colonies were isolated and expanded.

### 4. In Vitro Characterization

Clones producing high levels of GDNF were further characterized by their ability to deliver long-term GDNF expression in conventional cell culture and during encapsulation in vitro. Processing of GDNF from cell lines derived by the two different vector constructs were analyzed by GDNF Western blotting.

### 4.1. GDNF Release in Confluent 2D Cultures

Forty-seven GDNF clones were selected for long-term 2D culture test to assess morphology and GDNF release during 8 weeks of confluent culture. GDNF ELISA results from a selection of the best clones are shown in FIG. 2. The best clones produce up to 25 μg/ml/24 hrs in confluent cultures. In cultures where cells are passaged weekly the best clone (SBhoGDNF-125) produced 20 μg GDNF/105 cells/24 hrs for more than 6 months (results not shown). This is approximately 10-fold higher secretion levels compared to GDNF clones previously generated using non-optimized GDNF and standard transfection techniques.

4.2. Processing of GDNF from the Different Vector Constructs

Human GDNF cDNA encodes a 211 amino acid residue prepropeptide that is processed to yield a disulfide-linked dimeric glycoprotein. Mature GDNF is predicted to contain two 134 amino acid residue sub-units. The GDNF sequence contains two potential glycosylation sites. The predicted molecular weight (MW) of the un-glycosylated monomer is approximately 15.1 kDa.

To assess the processing of the GDNF secreted from clones derived by the two different vector constructs, one of each were selected for GDNF Western blot (WB) analyses. Conditioned medium from clones ARPE-19/pT2.CAn.ho.IgSP.GDNF #2 and ARPE-19/pT2.CAn.hoG #3 and were taken after 1 week in confluent 2D cultures. Samples from the CA-9 clone and recombinant GDNF from R&D system (Cat no. 212GD) were included as references.

GDNF WB results showed that GDNF produced from both of the clones were processed similarly as purified recombinant GDNF from R&D Systems. The GDNF protein secreted from the clones was predominantly present as glycosylated monomers and dimers of processed mature GDNF (FIG. 3). A smaller fraction of non-glycosylated GDNF (monomer and dimer) was also present. Pro-GDNF (predicted MW of non-glycosylated monomer: 21.6 kDa) was not detected.

4.3. GDNF Release from Devices

### 4.3.1. Encapsulation of Cells in EC Biodelivery Devices.

To improve device manufacturability, filling, viability and reproducibility, a new custom polysulfone (PS) membrane (Medivators, Plymouth, Minn.) and polyethylene terephthalate (PET) yarn scaffolding (Gloriana, Providence, R.I.) have been implemented. Devices were built with 7 mm long membranes fitted with yarn scaffold. Prior to filling, cells or parental RPE cells were cultured in growth medium. Cells were dissociated and suspended at a density of 12.500 cells/μl in HE-SFM (Invitrogen, Odense, DK). Four μl of cell solution (5×10⁴ cells in total) were injected into each device. Devices were kept in HE-SFM at 37° C. and 5% CO₂.

4.3.2. GDNF Secretion from Encapsulated Cell Clones In Vitro.

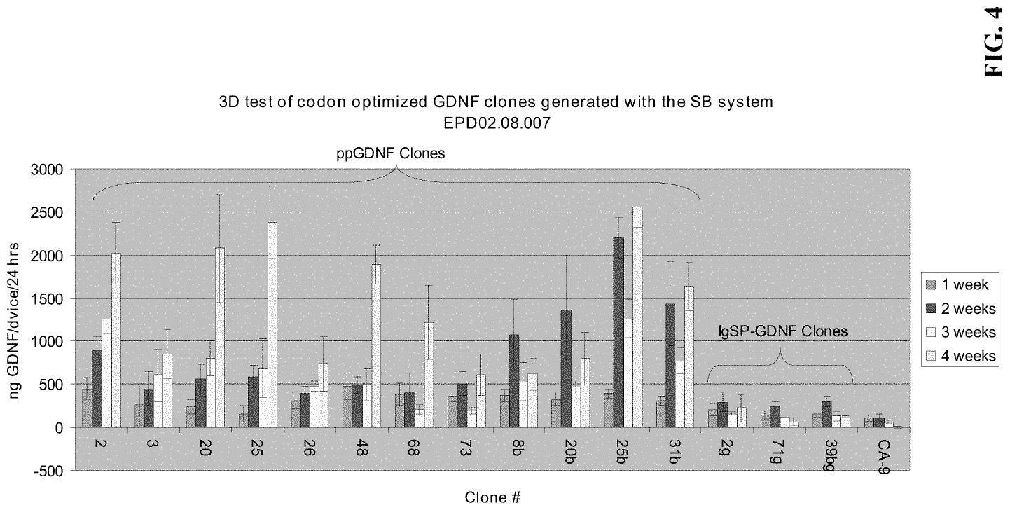

A total of 16 clones were analyzed in 3D culture (devices kept in vitro) over a period of 4 weeks. Each week, media samples (collected after 4 hours incubation) were taken from each device. The samples were frozen at −80° C. and all samples were analyzed by GDNF ELISA at the same time after the end of the 4 weeks. The results are shown in FIG. 4. Several clones perform equally well (2, 20, 25, 48, 68, and 25b). ppGDNF clones secrete significantly more GDNF than the IgSP-GDNF clones due to differences in the intra-cellular processing of the precursor molecules. The CA-9 cell line in FIG. 4 was created previously using non-optimized GDNF and standard transfection techniques. The best codon-optimized, Sleeping Beauty clones produce up to approximately 25-fold more than CA-9, showing the superior performance of clones using the two expression optimizing techniques in combination.

### 5. In Vivo Test of Selected Clones—2 Weeks

Nine clones were selected from the 3D test. Seven clones were derived from the pT2.CAn.hoG vector (ppG #2, 20, 25, 48, 68, 120 and 125) and two clones were derived from the pT2.CAn.ho.IgSP.GDNF vector (IgSP 2g and 39gb). All the clones, except the IgSP-GDNF clones, were producing >700 jig/ml after 4 weeks in 3D culture. The IgSP-GDNF clones (2g and 39gb) were included to test if this chimeric molecule, as opposed to the in vitro 3D test, would perform better than ppGDNF in vivo. A selection criterion was also cell morphology in the devices. Thus, high producer clones with different cell shapes and growth patterns were selected for the in vivo study.

### 5.1. Experimental Procedure

Devices filled with the nine new clones and CA-9 as reference were bilaterally implanted in rat striatum (n=30 rats) by an implantation cannula mounted to the stereotaxic frame (n=6 devices with each clone). The implantation coordinates with respect to Bregma were: AP: 0.0, ML: ±3.2, DV: −7.5, TB: −3.3.

After 2 weeks, rats were deeply anesthetized, decapitated and the brains removed. The devices were explanted and incubated at 37° C. in HE-SFM. Media samples (4 h incubation) for determination of GDNF release were collected the next day. GDNF concentrations in media samples were determined by GDNF ELISA. Devices were fixed in Formalin, embedded in Historesin and sectioned. Cell morphology was evaluated on Eosin and Hematoxylin (HE) stained device sections.

Tissue punches were taken around devices in fresh-frozen brains from three rats in each group. Homogenised tissue samples were analyzed by GDNF ELISA and GDNF Western blot.

The brains from the remaining three rats in each group were immersion fixed in Formalin solution for 48 h before cryoprotection in 30% (W/V) sucrose solution in 0.1 M sodium phosphate buffered saline (PBS). Brains were sectioned at the coronal level on a freezing microtome and sections were processed for GDNF immunohistochemistry.

5.2. GDNF Release Before Implantation and after Explantation

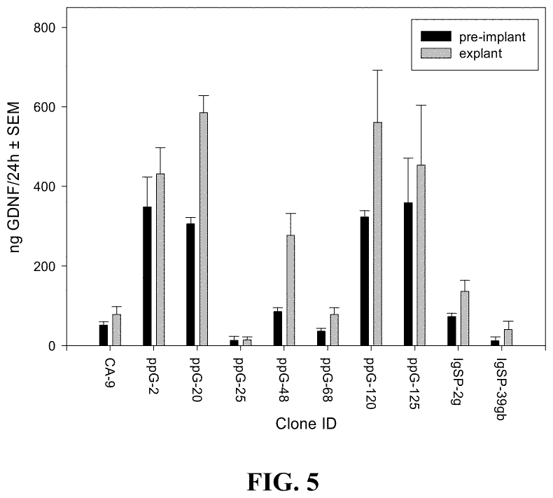

FIG. 5 shows the GDNF release from devices filled with the different clones measured in samples taken before implantation (2.5 weeks after filling) and the day following explantation after 2 weeks in rat brain. Four of the tested new clones, ppG-2, ppG-20, ppG-120 and ppG-125, showed markedly increased GDNF secretion (up to 7 fold increase) compared with CA-9.

### 5.3. HE Staining of Device Sections

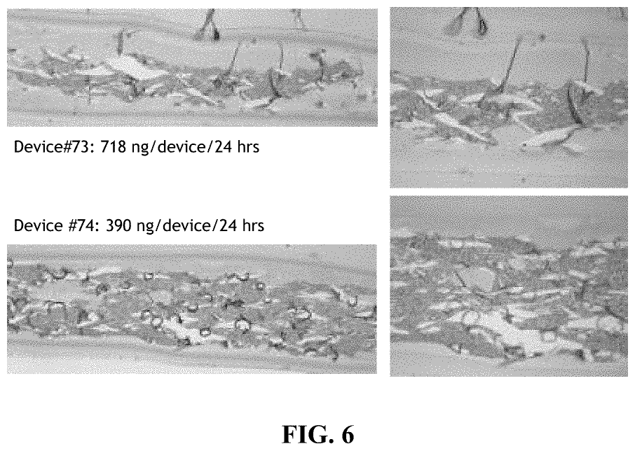

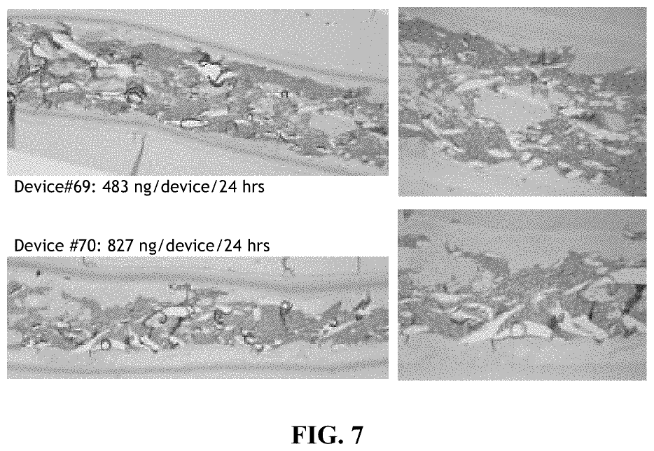

All of the GDNF producing clones showed a good cell survival in the devices after 2 weeks in rat brain. The cells were evenly distributed in the yarn scaffold throughout the devices and were not filled very densely. Representative HE stained sections for clone ppG-120 (FIG. 6) and ppG-125 (FIG. 7) are shown.

### 5.4. GDNF Tissue Levels, GDNF Immunohistochemistry

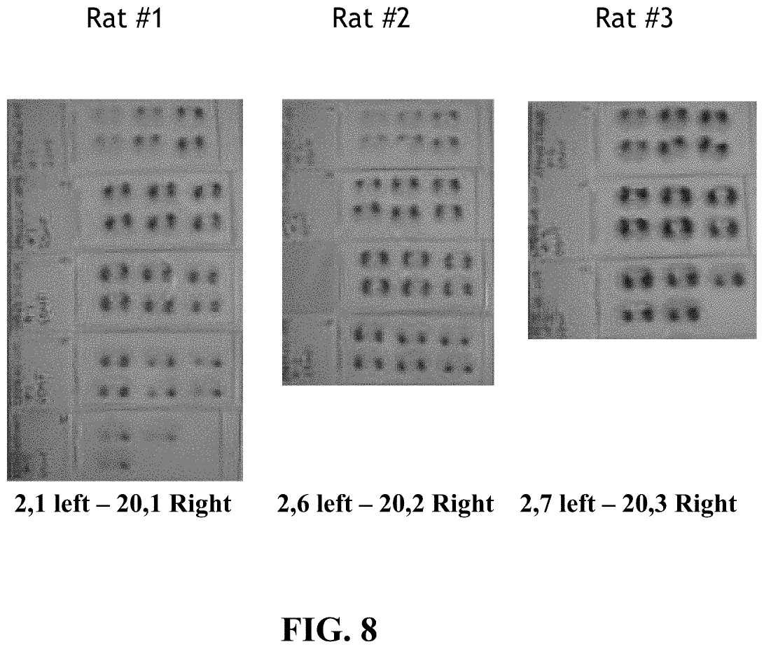

GDNF immunohistochemistry was performed on sections with implant sites for three devices of each GDNF producing clone. Results showed a prominent secretion of GDNF from the devices, covering all the striatum. Clones ppG-2, ppG-20, ppG-120 and ppG-125 showed particularly high GDNF tissue levels. The rat brain sections for clone ppG-2 and ppG-20 are shown in FIG. 8.

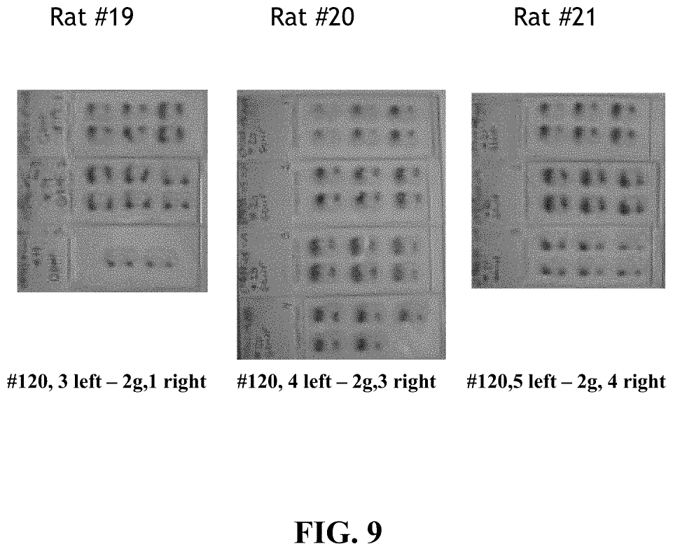

FIG. 9 shows the GDNF immunostainings for clone ppG-120 and IgSP-2g. In control sections from un-treated rats, no GDNF immunoreactivity was seen in striatum (data not shown).

### 5.5. GDNF Tissue Levels, GDNF ELISA

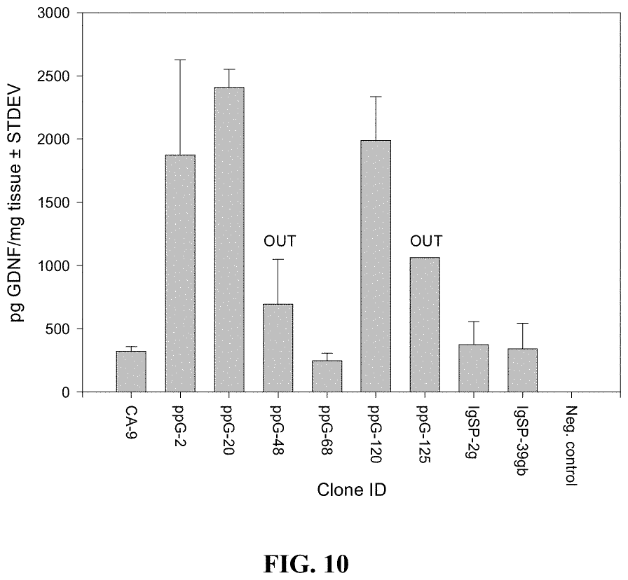

Tissue punches were taken around three devices from each clone, and homogenized tissue samples were analyzed by GDNF ELISA. Results in FIG. 10 show new clones with markedly higher tissue levels than CA-9 (at least a 6 fold increase), especially clones ppG-2, ppG-20, ppG-120 and ppG-125 showed high GDNF tissue levels. In addition, CA-9 shows a clearly improved performance than previously seen due to the replacement of the Akzo polyether sulfone membrane with polysulfone membrane (Medivators, Plymouth, Minn.) leading to increased GDNF release in the tissue. Altogether, GDNF tissue levels have increased from the range of around 20 pg/mg tissue previously seen for CA-9 to more than 2000 pg/mg tissue for the best clones.

### 5.6. Correct Processing of GDNF in Tissue

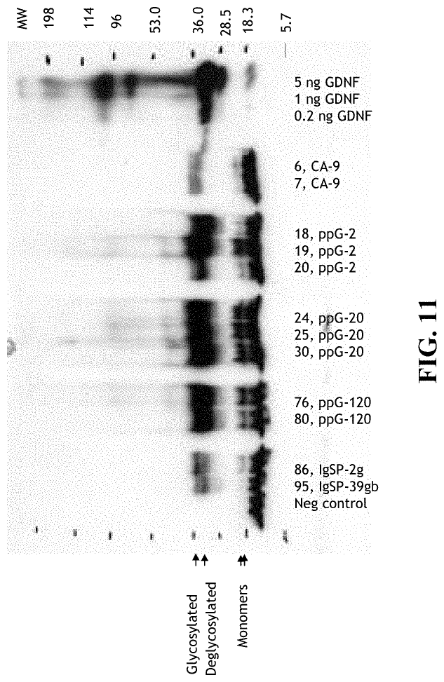

Selected homogenized tissue samples were analyzed by GDNF WB to examine the processing of the GDNF protein released in the brain. Homogenized tissue from the striatum of an untreated rat and purified recombinant GDNF (R&D Systems, Minneapolis, Minn.) were included as references. Monomers and dimers of glycosylated and non-glycosylated GDNF were observed (FIG. 11). No proGDNF was detected, showing that the GDNF secreted from the devices is correctly processed, also in the in vivo situation.

### 6. Test in the Rat 6-Hydroxydopamine (6-OHDA) Lesion Model

To determine whether EC biodelivery devices with the new clones released sufficient GDNF amounts to elicit a relevant biological effect, two clones were selected for test in the 6-hydroxydopamine (6-OHDA) rat striatal lesion model, which mimics some of the aspects of the dopaminergic (DA) cell death observed in Parkinson's disease. Clones ppG-2, ppG-20, ppG120 and ppG-125 were all potential candidates for testing. Clones ppG-120 and ppG-125 were chosen, as they appeared to have a slightly better survival after 2 weeks in vivo.

### 6.1. Experimental Procedure

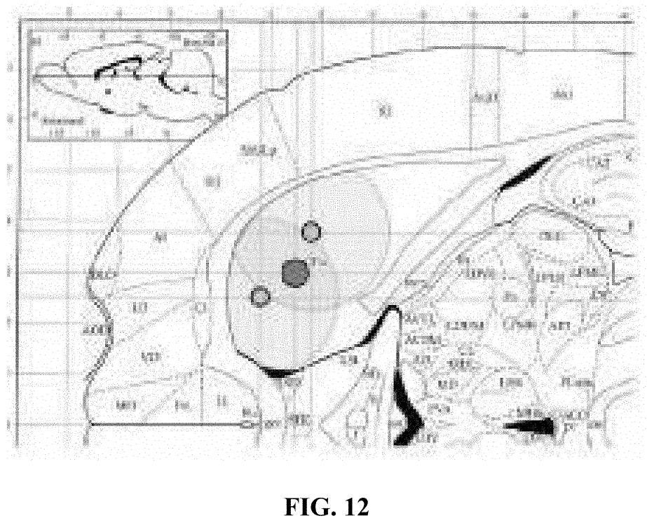

Devices filled with clone ppG-120, ppG-125 or without cells (n=10 for each) were implanted in the right striatum at the following coordinates with respect to Bregma: AP: 0.5, ML: 3.0, DV: −7.5, TB: −2.3. One week after device implantations, the 6-OHDA lesion was performed. Prior to surgery, rats were again anesthetized with Isofluran (Sigma-Aldrich, St. Louis, Mo.) and positioned in a stereotaxic frame. Two sites were injected with 10 μg 6-OHDA/site using a 28-gauge Hamilton syringe mounted to the stereotaxic frame at the following coordinates with respect to Bregma: (1) AP: 1.2; ML: 2.5, DV: −5.0, TB, −2.3 and (2) AP: 0.2; ML: 3.8, DV: −5.0, TB, −2.3. The 6-OHDA was infused in a total volume of 2 μl over 2 minutes. The injection cannula was left in place for an additional two minutes to allow the 6-OHDA to freely diffuse from the injection site. The cannula was then removed and the skin suture closed. FIG. 12 shows the placement of the device and the 6-OHDA injections.

After 6 weeks, rats were deeply anaesthetized, decapitated and the brains removed. The devices were explanted and incubated at 37° C. in HE-SFM. Media samples (4 h incubation) for determination of GDNF release were collected the next day. GDNF levels in media samples were determined by GDNF ELISA. De-vices were fixed in Formalin, embedded in Historesin and sectioned. Cell morphology was evaluated on HE stained device sections. The brains were immersion fixed in Formalin solution for 48 h before cryoprotection in 30% (W/V) sucrose solution in 0.1 M sodium PBS. Brains were sectioned at the coronal level on a freezing microtome and processed for GDNF and tyrosine hydroxylase (TH) immunohistochemistry.

6.2. GDNF Release Before Implantation and after Explantation

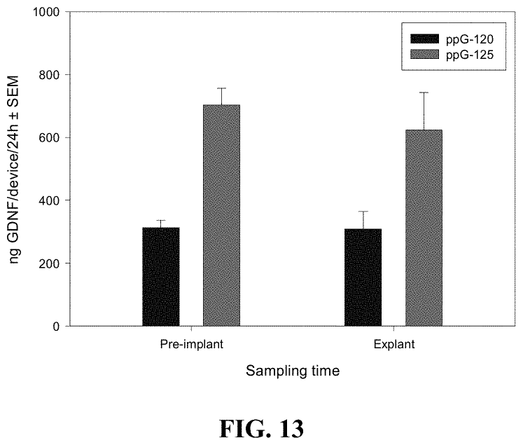

FIG. 13 shows the GDNF release from devices with clone ppG-120 and ppG-125, measured in samples taken before implantation (4 weeks after filling) and the day following explantation after test in the 6-OHDA lesion model (7 weeks in rat brain). Devices with clone ppG125 showed the highest GDNF release before implantation (703±53 ng GDNF/day) as well as after explantation (623±119 ng GDNF/day).

### 6.3. HE Staining, Device Sections

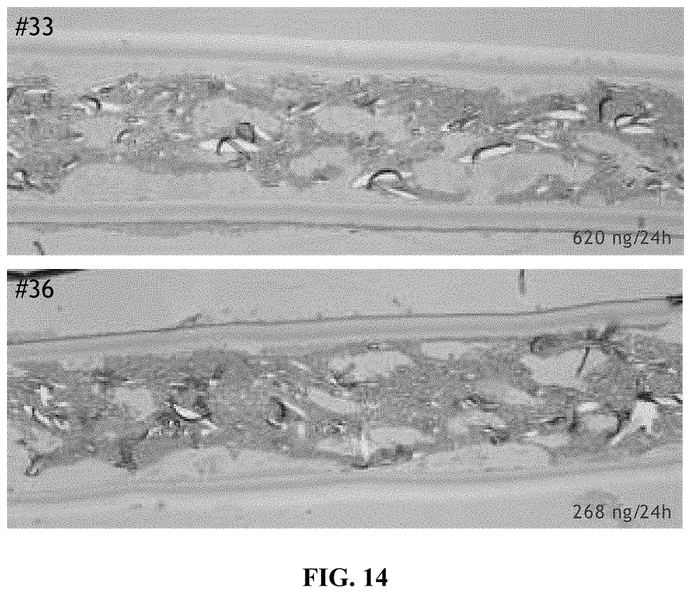

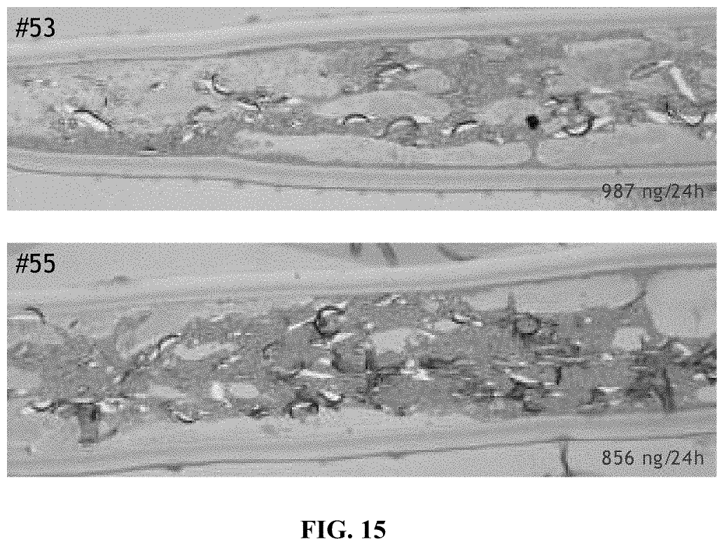

In general, the cells in the devices explanted after termination of the 6-OHDA experiment showed good survival. The cells were evenly distributed in the yarn scaffold throughout the devices and did not fill out the device very densely. Representative examples of HE stained sections with clone ppG-120 are shown in FIG. 14 and with clone ppG-125 in FIG. 15.

### 6.4. GDNF Tissue Levels, GDNF Immunohistochemistry

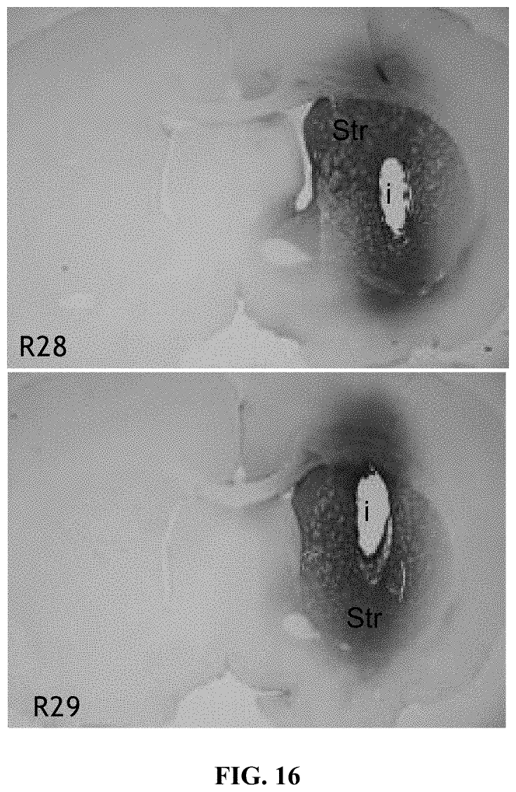

GDNF immunohistochemistry was performed on sections covering the striatum from all three experimental groups. The implant sites for empty control devices with no cells had no GDNF-immunoreactivity (data not shown). Rats implanted with devices filled with ppG-120 or ppG-125 cells showed a prominent secretion of GDNF from the devices, covering the striatum around the implant site. The GDNF-immunoreactivity generally appeared to be more pronounced around implant sites with clone ppG-125 than clone ppG-120. FIG. 16 shows representative examples of the GDNF diffusion from the devices with clone ppG-125.

### 6.5. Quantification of Neuroprotective Effect of GDNF Clones

### 6.5.1. Image Analyses of TH-Immunoreactive Area

To assess the toxic effect of 6-OHDA as well as the protective effect of GDNF on the DA neurons, immunohistochemistry using an antibody against the marker for DA neurons, tyrosine hydroxylase (TH) was performed on brain sections covering the striatum as well substantia nigra (SN) from all three experimental groups.

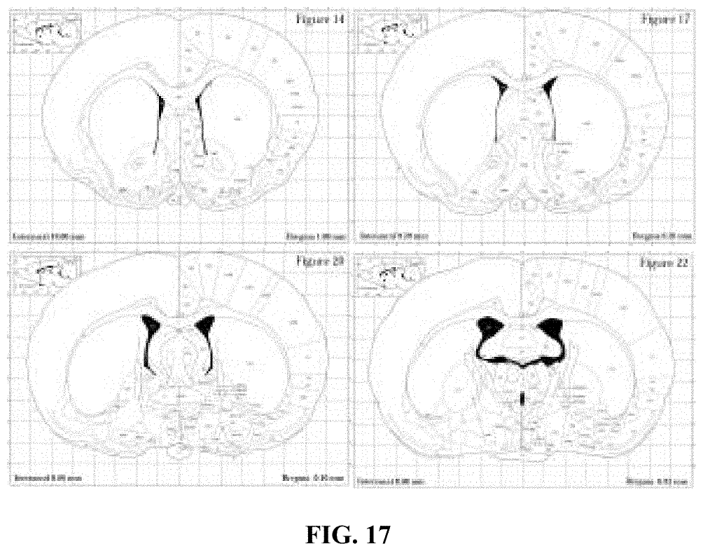

To evaluate the size of the striatal 6-OHDA lesion, image analyses was performed on four selected sections in each rat (FIG. 17). Digitized images were taken using an Olympus® BX61 microscope (Olympus Corporation of the Americas, Center Valley, Pa.), and analysis on the size of the area with TH-immunoreactivity was subsequently performed using the VisioMorph software (Visiopham, Copenhagen, DK). Likewise, in SN with the DA cell bodies projecting to the striatum, three defined sections were selected for analyses of the TH-immunoreactive area. A protective effect of GDNF should manifest in an increase the number of surviving DA neurons in SN, while an effect of regeneration (sprouting) of damaged DA projecting fibers in striatum would not be expected at the evaluated time point, 6 weeks after the lesion. In accordance with this, no sprouting of TH-immunoreactive fibers was observed in the lesioned area in striatum in either of the groups. The size of the striatal 6-OHDA lesion in the current study is therefore anticipated to be independent of the treatment.

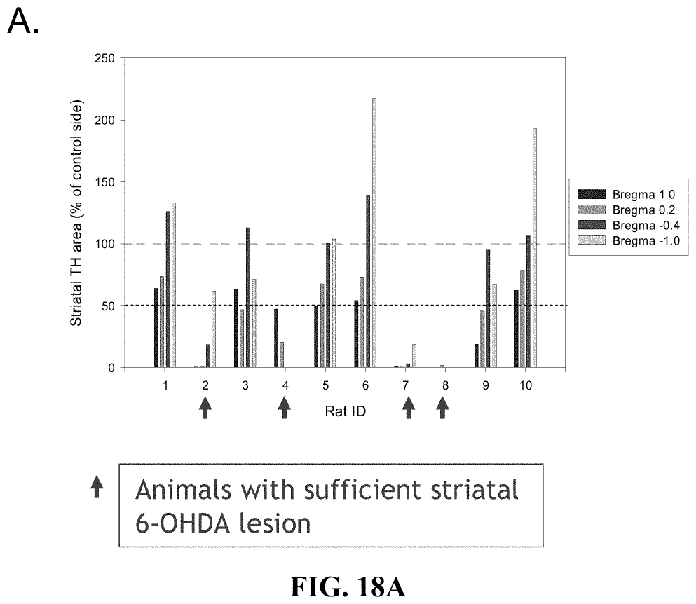

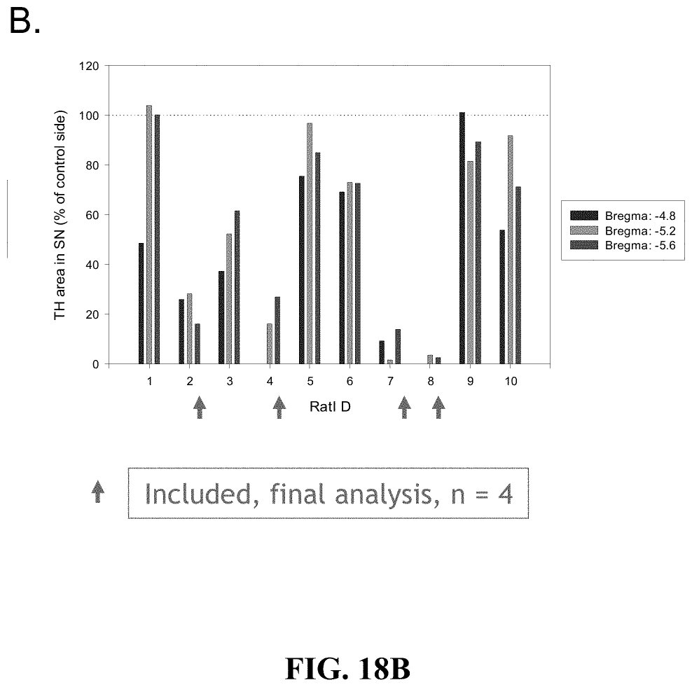

FIG. 18 shows the results for the image analyses of the individual rats in the control group with empty devices for striatum (A) and SN (B). Some of the animals did not show a marked decrease in the striatal TH immunoreactivity, indicating that the 6-OHDA induced lesion was relatively small in these animals. In accordance with this, the TH-immunoreactive area in SN was also relatively high. To have a sufficient window to detect a protective effect of GDNF, the animals should have a sufficient striatal 6-OHDA lesion (less than 50% TH immunoreactivity, compared with control side) to be included in the final analyses. Four animals in the control group fulfilled this criterion (indicated by arrows in FIG. 18).

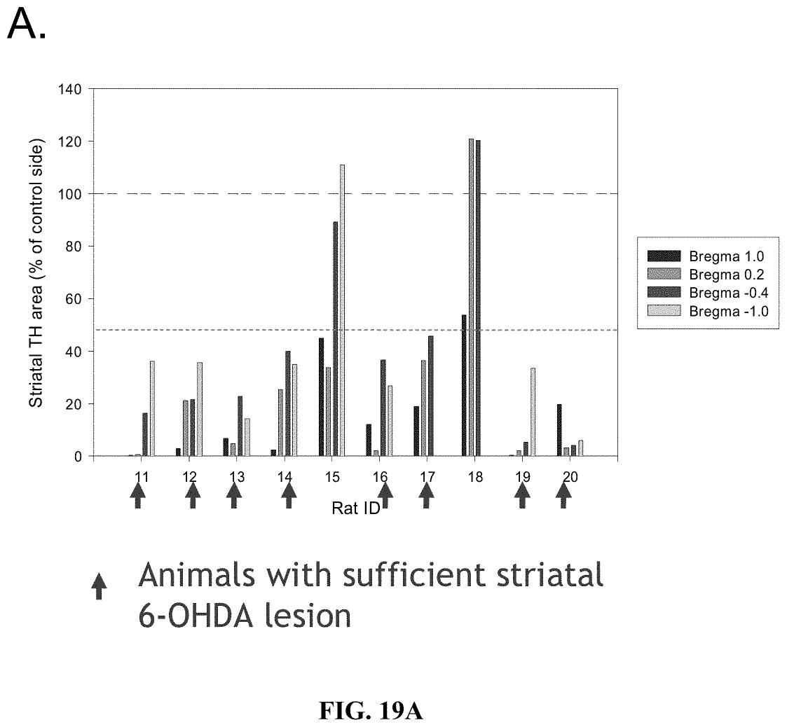

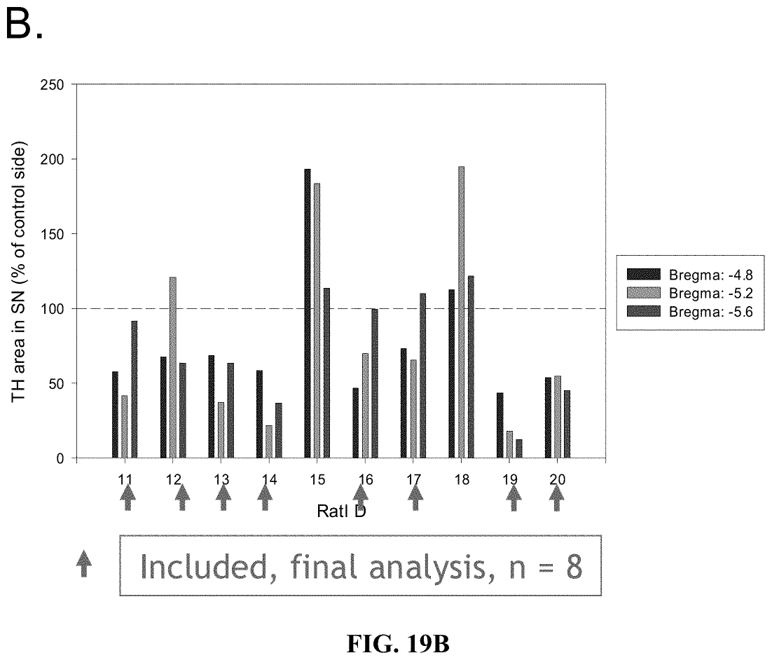

In the group implanted with devices containing ppG-120 cells, most of the rats (n=8) had a striatal 6-OHDA lesion corresponding to less than 50% of the control side TH immunoreactivity (FIG. 19). Rats nos. 15 and 19 had insufficient striatal lesion and were therefore excluded from the final evaluation.

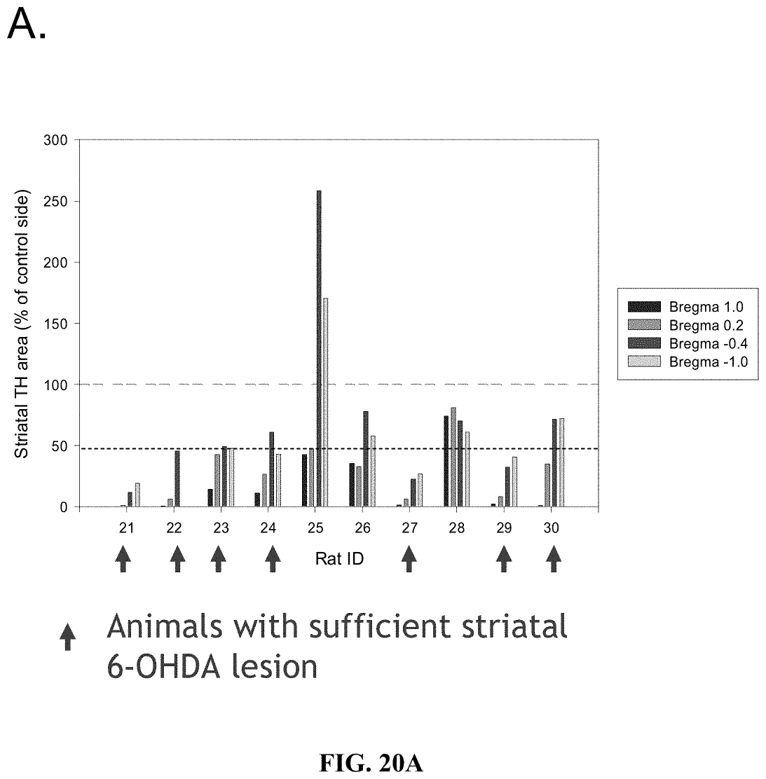

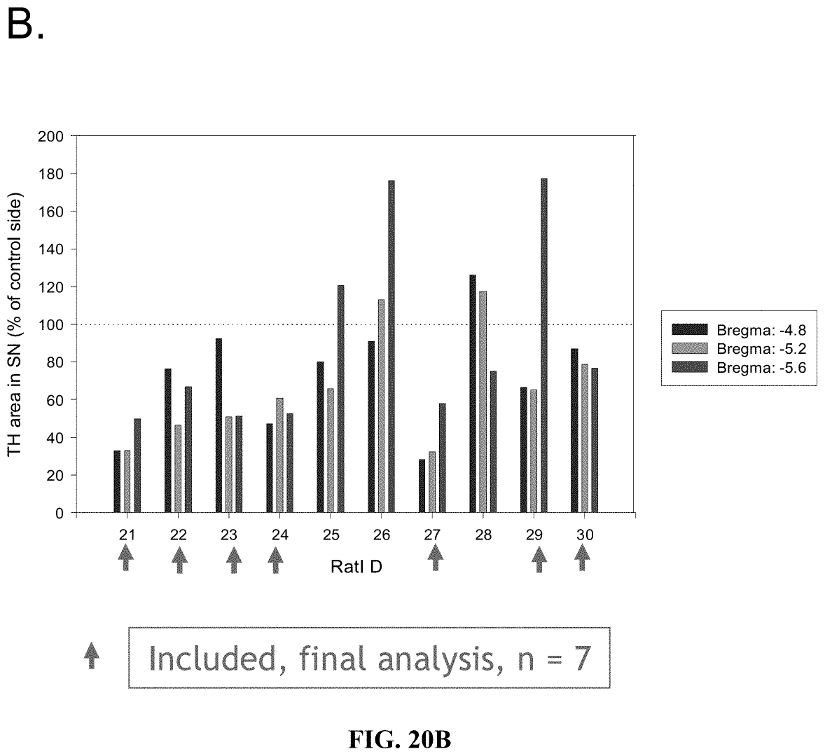

In the group implanted with devices containing ppG-125 cells, seven out of ten rats had a striatal 6-OHDA lesion corresponding to less than 50% of the control side TH immunoreactivity (FIG. 20).

In the two groups with devices having GDNF secreting cells, the lesions are generally more efficient, so the presence of the implanted device per se is unlikely to be the cause. A protective effect of the empty devices is not probable, and as previously mentioned no sprouting of TH-immunoreactive fibers was observed in the lesioned area in striatum in either of the groups. Small deviations in the handling and injections of the relatively unstable 6-OHDA might have caused the variations.

### 6.5.2. Result, Image Analyses of TH-Immunoreactive Area

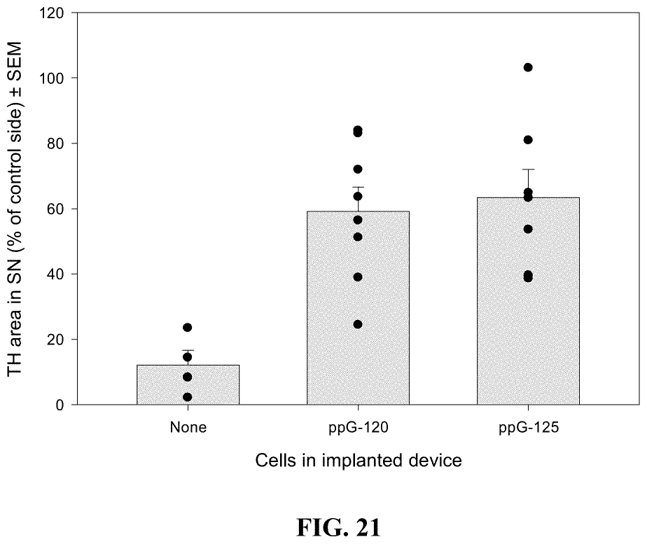

FIG. 21 shows the mean percentage of surviving TH-positive neurons (corresponding to the TH-immunoreactive area) in the lesioned SN. A significant protective effect of both GDNF secreting clones was found (one way ANOVA followed by multiple comparisons versus control group (Dunnett's Method), p<0.05).

### 6.5.3. Countings of TH-Positive Cells in SN

In addition to the image analyses, manual cell countings on TH-positive neurons were performed on the three selected sections from substantia nigra, as previously described by Sauer et al. (1995), Proc Natl Acad Sci USA, 92(19):8935-9.

### 6.5.4. Result, Countings of TH-Positive Cells in SN

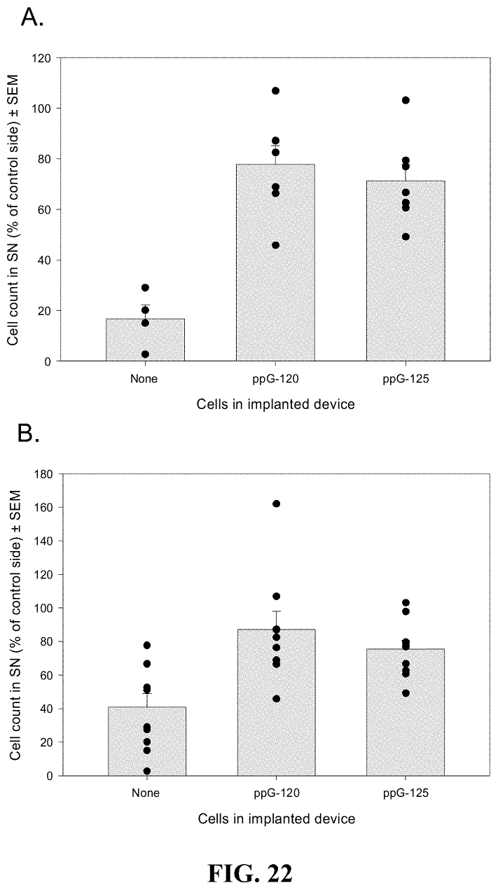

FIG. 22 shows the results of the manual cell countings in SN. As described in section 6.5.1, animals with small striatal 6-OHDA lesions (TH-immunoreactive area in the lesion side >50% of that in the control side) were excluded from the final evaluation (FIG. 22A). Results confirmed a significant neuroprotective effect on the DA neurons of devices with ppG-120 as well as ppG-125 (Significant difference from control group with empty devices, One way ANOVA followed by all Pairwise Multiple Comparison Procedures, Tukey Test, P<0.05). There was no significant difference in the effect of ppG-120 and ppG-125.

When including the cell countings from all the animals, there was still a significant difference (One way ANOVA followed by Tukey Test, P<0.05) between the control group and the two GDNF producing clones (FIG. 22B).

The manual cell countings showed the same picture as the image analyses, but with a slightly higher protective effect of the clones, ppG120 (78±7% vs. 59±7%) and ppG125 (71±5% vs. 63±9%). In the control group there was less difference between the two evaluation methods (17±6% vs. 12±4%). GDNF has previously been shown to down regulate TH expression (Georgievska et al., (2002), Exp Neurol, 177(2):461-74 and Georgievska et al., (2004), J Neurosci, 24(29):6437-45), and this effect was also evident in the current study (FIG. 23). The manual countings of TH-immunoreactive cells might therefore be more accurate than the image analyses on area.

In addition, immunostainings using an antibody against the vesicular monoamine transporter (VMAT), which is also expressed by DA neurons and not down-regulated by GDNF, could be performed. That might lead to a picture showing an even better effect of the clones.

### 6.5.5. Conclusions

The two GDNF producing clones, ppG-120 and ppG-125, showed comparable neuroprotective effects. As clone ppG-125 released the highest levels of GDNF before implantation, after explantation and in the tissue, it is chosen as the primary clinical cell candidate for further development.

### 7. Long-Term (6 Months) GDNF Delivery in Guinea Pig Cochlea

An in vivo study to confirm the function and safety of devices secreting GDNF following implantation into guinea pig cochlea was conducted. A total of eighteen (18) guinea pigs were used. Each animal received a unilateral implant of a device loaded with GDNF-secreting cells. The control, non-implanted cochlea was used as a control. The overall in vivo study duration was six (6) months. GDNF release from the encapsulated cells was quantified from all devices prior to implantation and after explantation 1, 3, 4, 5, and 6 months later. After explantation, the cochlea from selected animals (timepoints 3-6 months) were processed for histopathological analyses.

7.1 Device manufacture: Cells were plated and maintained at 37° C., 90% humidity and 5% CO₂ in T-175 flasks with growth medium; DMEM+glutamax (lx) supplemented with 10% fetal bovine serum. Routine culture will consist of feeding the cells every 2-3 days and passaging them at 7075% confluence. Cells were encapsulated into 3 mm long hollow fiber membranes manufactured from an in-house manufactured polysulfone membrane internally fitted with polyethylene terephthalate yarn scaffolding. Devices were kept in HE-SFM at 37° C. and 5% CO2 for approximately 2 weeks prior to surgical implantation.

7.2 Surgical Implantation: Prior to surgery, all animals were anesthetized using Ketamine (40 mg/kg) and xylazine (5 mg/kg) IM. The hair was clipped on the left side from the head and along the ear and the surgical site aseptically washed with betadine and alcohol. The animal was draped with a sterile towel leaving only the surgical site exposed. The animals were monitored by the surgeon and the ACF veterinary staff for suitable hemostasis and respiration.

An incision (approximately 1.0 cm) was made proximal to the back of the left ear to expose the temporal bone. The overlying galea and fascia was pushed to the side and a 2 mm hole was made, using a #11 blade, in the temporal bone overlying the cochlea. Using a surgical microscope, a small (1 mm) cochleostomy was made in the side of the cochlea approximately 1 mm from the round window using a 0.5 mm diameter fine tipped, hand-held drill. A single device (0.4 mm

diameter×3.0 mm length) was placed into the cochlea with only the proximal tip remaining at the entry to the cochlea to allow subsequent retrieval. A piece of gel-foam was placed over the outer mastoidotomy and the skin was closed using Vicryl sutures.

7.3 Histopatholgy: At each specified time-point for device removal, animals were anesthetized as described above, prepared for surgery, and the previous implant was visualized. After retrieval, animals were sacrificed using overdose pentobarbital euthanasia followed by decapitation. Heads wee immersion fixed in 10% formalin prior to histological processing at CBSET Laboratories (Lexington, Mass.). Both cochlea from each head were explanted, decalcified, processed, and embedded in paraffin to produce a total of thirty (30) tissue blocks. Blocks were step cut, resulting in approximately ten (10) sections per cochlea (n=300 slides total). Sections were mounted on slides and stained with hematoxylin and eosin (H&E) (Celnovte Biotechnology, Rockville, Md.).

Three (3) slides from each cochlea (one (1) from each proximal, mid, and distal areas of the cochlea) were assessed by a board-certified veterinary pathologist according to Table 1 below. Assessed parameters included inflammation, injury, and nerve loss. The Study Pathologist was blinded to the treatment matrix at the time of the pathologist read.

TABLE 1
Microscopic Scoring Matrix

| Score | Microscopic Changes Scoring Matrix |
|---|---|
| 0 | No observable change |
| 1 | Minimal - a nearly imperceptible feature/change in the tissue |
| 2 | Mild/moderate - an easily identifiable and/or notable feature/ |
| | change in the tissue |
| 3 | Marked/severe - prominent to overwhelming feature/change in the |
| | tissue |

7.4 Observations: Animals were routinely observed for behavior and health status. All animals recovered rapidly post-surgery and demonstrated normal activity and feeding behavior within several hours post-recovery. There were no gross detrimental effects of continued GDNF secretion on behavior.

All devices were easily retrieved intact and without tissue adherence to the membrane. Prior to implantation devices secreted approximately 32.4 ng GDNF/24 hours. When examined post explant the average daily secretion of GDNF was increased with all devices continuing to have high and quantifiable levels of GDNF. Device secretion was relatively stable with peak output between 4-5 months. Individual device output is shown in Table 2 below.

7.5 Histopathology: A total of fifteen (15) animals from the three to six (3-6) month time-points were used for histopathological analyses. In this guinea pig model of cochlear implants, histologic parameters of inflammation, fibrosis, and injury were minimal to moderate, increased slightly over time by five (5) months, decreased slightly at six (6) months and were restricted to the area immediately surrounding the implant. Injury to the nerve was rare a nearly imperceptible when present and was observed only at three (3) and four (4) months; by the later time points, no nerve injury was observed. Necrosis was not a feature of any of the treated cochleae.

TABLE 2
Long-term GDNF secretion from devices
Implanted Into Guinea Pig Cochlea

| | Guinea | | Pre-implant | Post explant | |
|---|---|---|---|---|---|
| Time in | Pig | | GDNF | GDNF | Histo- |
| vivo | # | Device # | (ng/ml/day) | (ng/ml/day) | pathology |
| 1 month | 1 | CD-1 | 18 | 350 | No |
| 1 month | 2 | CD-2 | 29 | 287 | No |
| 1 month | 12 | CD-12 | 24 | 670 | No |
| 3 months | 3 | CD-3 | 31 | 324 | Yes |
| 3 months | 4 | CD-4 | 33 | 431 | Yes |
| 3 months | 6 | CD-5 | 27 | 203 | Yes |
| 3 months | 7 | CD-7 | 25 | 437 | Yes |
| 3 months | 8 | CD-8 | 44 | 356 | Yes |
| 3 months | 9 | CD-9 | 50 | 268 | Yes |
| 4 months | 10 | CD-10 | 31 | 1623 | Yes |
| 4 months | 11 | CD-11 | 34 | 266 | Yes |
| 4 months | 14 | CD-14 | 5 | 1083 | Yes |
| 5 months | 15 | CD-15 | 32 | 673 | Yes |
| 5 months | 16 | CD-16 | 54 | 726 | Yes |
| 5 months | 17 | CD-17 | 44 | (1383)* | Yes |
| 6 months | 18 | CD-18 | 29 | 567 | Yes |
| 6 months | 20 | CD-20 | 41 | 522 | Yes |
| 6 months | 22 | CD-22 | 32 | 558 | Yes |

*Measured on day 1 of explant but device was lost for day 2 pulse used for other values

7.6 Conclusions: 1). GDNF was continuously secreted from all devices (ranging from 1-6 months) when implanted into the guinea pig cochlea; 2). all devices were easily retrieved intact and without tissue adherence to the membrane; and 3). histopathological analyses confirmed that device implantation and long-term GDNF secretion produced only minimal to moderate inflammation and fibrosis. When observed, these parameters were localized to the implant site and are consistent with changes observed with medical device insertion in the cochlea in general.

8. Histopathological Evaluation of Cochlear Implants in Guinea Pigs

8.1. Procedure: Fifteen (15) formalin-fixed guinea pig heads were collected from animals that underwent unilateral implantation of cochlear devices. Heads were collected at the following time points post-implantation: three (3) months (n=6), four (4) months (n=3), five (5) months (n=3), and six (6) months (n=3). Both cochlea from each head were explanted, decalcified, processed, and embedded in paraffin to produce a total of thirty (30) tissue blocks. Blocks were step cut, resulting in approximately ten (10) sections per cochlea (n=300 slides total). Sections were mounted on slides and stained with hematoxylin and eosin (H&E). Three (3) slides from each cochlea (one (1) from each proximal, mid, and distal areas of the cochlea) were assessed by a board-certified veterinary pathologist according to Table 1. Assessed parameters included inflammation, injury, and nerve loss.

TABLE 1
Microscopic Scoring Matrix

| Score | Microscopic Changes Scoring Matrix |
|---|---|
| 0 | No observable change |
| 1 | Minimal - a nearly imperceptible feature/change in the tissue |
| 2 | Mild/moderate - an easily identifiable and/or notable feature/ |
| | change in the tissue |
| 3 | Marked/severe - prominent to overwhelming feature/change in the |
| | tissue |

8.2 Data Acquisition and Analysis: Values and/or observations obtained from the histomorphologic analysis were entered into a Microsoft Excel spreadsheet. Ordinal histologic data (scores) were reported as the group median and mean+SD. Additional data analysis were performed as deemed necessary (e.g., for comparative or clarification purposes). Calculations, data organization and the graphs were generated using Microsoft® Excel® software (Version 14).

8.3 Histomorphology: Serial sections through the treated left cochlea consistently showed response to implant presence in the form of minimal to mild/moderate localized inflammation and fibrosis, and the expected surgical disruption of the surrounding bony trabeculae. Sections from 3 out of 15 (20%) treated cochleae showed that the cochlear nerve was slightly affected by these changes, at 3-4 months. Untreated right sided cochleae were uniformly unremarkable (not displayed graphically).

8.3.1 Pertinent Histomorphological Observations: Treated left cochleae showed minimal to mild/moderate histological changes related to implant presence, including inflammation, injury, and host tissue fibrotic repair response. The treated area showed a zone of collagenous fibrous connective tissue around the implant tract, surrounded by a chronic, localized inflammatory cell infiltrate predominated by lymphocytes, and minor disruption of the surrounding bone. Two treated cochleae at the three-month time point (Animals 3 and 8; 33% incidence) and one at the four-month time point (Animal 11; 33% incidence) showed minimal nerve injury in the form of axonal loss, faint myelin sheath swelling, or rare digestion chamber formation. This injury was nearly imperceptible and was present in one or two out of the three scored sections of the cochleae from those animals. Inflammation, injury, and fibrosis showed a slight increase over time, peaking at five (5) months and then decreasing slightly at six (6) months. Detectable nerve injury was present only at three (3) and four (4) months. Necrosis was not a feature at any time point. Pertinent histomorphological observations scores for the left cochlea are summarized in Tables 2-4.

TABLE 2
Mean ± SD, Median and Incidence of Pertinent Histomorphology Observations - Left Side

| | 3 Months | | 4 Months | | 5 Months | | 6 Months | |
|---|---|---|---|---|---|---|---|---|
| Parameter | (n = 6) | | (n = 3) | | (n = 3) | | (n = 3) | |
| Inflammation | 1.39 ± 0.65 | 100% | 1.67 ± 0.58 | 100% | 1.78 ± 0.19 | 100% | 1.67 ± 0.34 | 100% |
| | 1.50 | | 2.00 | | 1.67 | | 1.67 | |
| Injury | 0.78 ± 0.62 | 67% | 1.33 ± 0.34 | 100% | 1.89 ± 0.19 | 100% | 1.44 ± 0.51 | 100% |
| | 1.00 | | 1.33 | | 2.00 | | 1.33 | |
| Nerve Loss | 0.17 ± 0.28 | 33% | 0.11 ± 0.19 | 33% | 0.00 ± 0.00 | 0% | 0.00 ± 0.00 | 0% |
| | 0.00 | | 0.00 | | 0.00 | | 0.00 | |
| Fibrosis | 1.28 ± 1.00 | 67% | 1.44 ± 0.51 | 100% | 1.78 ± 0.19 | 100% | 1.45 ± 0.69 | 100% |
| | 1.84 | | 1.33 | | 1.67 | | 1.67 | |
| Necrosis | 0.00 ± 0.00 | 0% | 0.00 ± 0.00 | 0% | 0.00 ± 0.00 | 0% | 0.00 ± 0.00 | 0% |
| | 0.00 | | 0.00 | | 0.00 | | 0.00 | |

Microscopic Changes Scoring Matrix: 0 = No observable change; 1 = Minimal - a nearly imperceptible feature/change in the tissue; 2 = Mild/moderate - an easily identifiable and/or notable feature/change in the tissue; 3 = Marked/severe - prominent to overwhelming feature/change in the tissue.

8.3.2 Observations: The surgical placement and chronic indwelling of intracochlear devices containing GDNF secreting human cells were consistent with overall biocompatible profile without adverse effects. Histologic evidence of inflammation, fibrosis, and injury was present only in treated ears, restricted to the area immediately surrounding the implant and at a level generally anticipated with chronic presence of biocompatible implant. Responses at the three (3) month time point were absent to minimal in 2/6 animals and minimal to moderate in 4/6 animals. Responses at four (4) months were absent to moderate in ⅔ animals and minimal to moderate in ⅓ animals. Responses were minimal to moderate in ⅔ animals and moderate in ⅓ animals at five (5) months and at six (6) months were absent to moderate in ⅓ animals, minimal to moderate in ⅓ animals, and moderate in ⅓ animals. Injury to the nerve was rare a nearly imperceptible when present and was observed only at three (3) and four (4) months; by the later time points, nerve injury was not a feature. Necrosis was not a feature of any of the treated cochleae.

### EQUIVALENTS

While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

Unless otherwise indicated, all numbers expressed quantities of ingredients, reaction conditions, and so forth use in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention.

The above discussion is meant to be illustrative of the principle and various embodiments of the present invention. Numerous variations, combinations and modifications will become apparent to those skilled in the art once the above disclosure is fully appreciated. It is intended that the following claims be interpreted to embrace all such variations and modifications.

I claim:

1. A mammalian cell capable of secreting glial derived neurotrophic factor (GDNF) or a functional equivalent thereof in amounts in excess of 20 μg GDNF/10⁵ cells/24 hours.

2. The cell according to claim 1, wherein said mammalian cell is selected from the group consisting of ARPE-19 cells, CHO cells, BHK cells, R1.1 cells, COS cells, HEK293 cells, PC12 cells, HiB5 cells, RN33b, neuronal cells, fetal cells, MDX12 cells, C2C12 cells, HeLa cells, HepG2 cells, striatal cells, neurons, astrocytes, interneurons, killer cells, helper T-cells, cytotoxic T-lymphocytes and macrophages.

3. The cell according to claim 1, wherein said mammalian cell is selected from the group of mammals consisting of mouse, rat, rabbit, dog, cat, pig, monkey, and human.

4. The cell according to claim 3, wherein said mammalian cell is a human cell.

5. The mammalian cell according to any of the claims 1 to 4, wherein said mammalian cell is capable of being attached to a support matrix.

6. A method of producing glial derived neurotrophic factor or a functional equivalent thereof, said method comprising the steps of:
i. culturing the cell of any of the claims 1 to 4 in a medium wherein said cell secreted glial derived neurotrophic factor; and
ii. recovering the secreted glial derived neurotrophic factor from the culture medium.

7. An implantable cell culture device, the device comprising:
isolated host cells as defined in any of claims 1 to 4; and
ii. a semi-permeable membrane permitting the diffusion of a growth factor secreted from said isolated cell line situated within said device through said membrane.

8. The device according to claim 7, wherein the semi-permeable membrane is immunoisolatory.

9. The device according to claim 7, wherein the device further comprises a matrix disposed within the semi-permeable membrane.

10. The device according to claim 7, wherein the device is capable of secreting glial derived neurotrophic factor in excess of 10 ng of biologically active glial derived neurotrophic factor per 24 hours, preferably in excess of 20 ng of biologically active glial derived neurotrophic factor, more preferably in excess of 40 ng/24 hours, more preferably in excess of 60 ng/24 hours.

11. The device according to claim 7, wherein the device further comprises a tether anchor.

12. The cells according to any of claims 1 to 4 for use in a method of treating a nervous system disease, said method comprising transplanting to an individual in need thereof a therapeutically effective amount of the cells of any of claims 1 to 4.

13. The cells according to claim 12, wherein said disease of the nervous system disease is Parkinson's Disease.

14. The device according to claim 7, wherein said device is implanted into the cochlea of a patient in need of treatment.
